(19) United States
(12) Patent Application Publication
(10) Pub. No.: US 2018/0159468 A1
(43) Pub. Date: Jun. 7, 2018

(54) DETERMINING THE CONDITION OF PHOTOVOLTAIC MODULES

(71) Applicant: BT Imaging Pty Ltd, Redfern (AU)

(72) Inventors: Thorsten TRUPKE, Randwick (AU); Ian Andrew MAXWELL, Redfern (AU); Robert Andrew BARDOS, Kingsford (AU); Juergen WEBER, Maroubra (AU)

(21) Appl. No.: 15/366,013

(22) Filed: Dec. 1, 2016

(51) Int. Cl. H02S 50/15; G01N 21/66; G01N 21/64; G01N 21/88; G01N 33/00

(52) U.S. Cl. CPC H02S 50/15; G01N 21/66; G01N 2021/8887; G01N 21/8851; G01N 33/00; G01N 21/6456

(57) ABSTRACT

Some examples include determining the condition of photovoltaic modules at one or more points in time, in particular using line-scanning luminescence imaging techniques. One or more photoluminescence and/or electroluminescence images of a module may be acquired and processed using one or more algorithms to provide module data, including the detection of defects that may cause or may have caused module failure. Additionally, some examples include determining the condition of photovoltaic modules, such as throughout the production, transport, installation and service life of the photovoltaic modules.

### FIELD OF THE INVENTION

The present invention relates to apparatus and methods for determining conditions of photovoltaic modules, in particular using luminescence imaging techniques. Some implementations of the present invention have been developed for use in inspecting or otherwise determining conditions of photovoltaic modules comprising silicon photovoltaic cells, and are described with reference to this application. However it will be appreciated that the present invention is not limited to this particular field of use.

### BACKGROUND OF THE INVENTION

Any discussion of the prior art throughout this specification should in no way be considered as an admission that such prior art is widely known or forms part of the common general knowledge in the field.

Photovoltaic modules (hereafter ‘module’ or ‘modules’) are becoming an increasingly significant part of the global power generation mix. It is estimated that there are more than a billion modules currently installed worldwide, a figure that is growing by 10 to 20% per annum. The majority of installed modules contain a rectangular array of sixty or seventy-two monocrystalline or multicrystalline silicon photovoltaic cells (hereafter ‘cell’ or ‘cells’), although modules based on thin film materials such as cadmium telluride, copper indium gallium selenide (CIGS) or amorphous silicon are also relatively common as are modules with larger or smaller numbers of silicon cells. FIG. 1 shows in schematic plan view a typical module 100 comprising a rectangular array of sixty silicon cells 102 wired as three strings 104 of twenty cells connected in series, and with electrical contacts 106 for extracting the charge carriers generated by absorption of solar radiation (or similar) in the cells. Each string 104 has a by-pass diode 108 connected in parallel to limit the extended influence of defective or temporarily shaded cells. With sixty 150×150 mm cells arranged in a six-by-ten close-packed rectangular grid a module 100 will have a total width 110 of about 1.0 m and a total length 112 of about 1.65 m. As shown in schematic plan view in FIG. 2, a thin film module 200 typically comprises an array of narrow strip-shaped cells 202 connected in series, with electrical contacts 106 at each end. Thin film modules are typically formed in a wide range of sizes by depositing doped semiconductor materials using thin film deposition techniques on a substrate 204 such as glass coated with a transparent conductive oxide, with cell structure usually created using laser scribing techniques.

Modules are typically intended to have an operational life of around twenty or twenty five years, with warranties typically covering those time scales. However there are several failure modes that can compromise the performance not only of individual cells within a module, but also of surrounding cells or even an entire module. Some failure modes can also cause hot spots, with an associated risk of fire or further damage to the module. It has been claimed that in some cases up to 10% of modules in an installation will fail during their warrantied lifetime, representing a large commercial problem. ‘Failure’ of a module can be an outright fail where no power is generated, or a drop in power generation to below the warrantied level, usually calculated according to a formula that allows for a fixed percentage drop per annum.

Examples of failure modes for individual cells include cracks, shunts and localised regions of excessive series resistance that may be associated with breaks in the metal contact pattern or poor contact between the metal pattern and the silicon or other cell material. Breaks in the electrical connections between cells can also fully or partially isolate one or more cells in a module. Such failure modes may be induced for example by cell or module manufacturing errors, or by improper handling during module transport or installation. They may also be initiated and/or grow over months and years in the field, e.g. by ingress of water and oxygen, or the inevitable thermal cycling and UV degradation of organic materials in the module. Cracks are a particularly insidious failure mode because of their propensity to grow over time. For example a small crack in a cell initiated during module manufacture or shipping may have no discernible effect on performance at the time of module installation, but can grow because of thermal cycling or other environmental stress for example. Various so-called light-induced degradation mechanisms are known, which decrease the electrical performance of an illuminated module over time upon illumination. A number of physical mechanisms for this degradation have been identified, involving for example the Boron-Oxygen defect prevalent in monocrystalline silicon cells. Another degradation mechanism is potential-induced degradation, which is the result of large voltage differences between the cells and the glass surface and frame of a module. Yet another possible degradation mechanism is oxidation-induced cloudiness of the ethylene vinyl acetate (EVA) polymer typically used to encapsulate silicon cells within a module.

It is therefore desirable, especially for warranty purposes, to be able to inspect or determine the condition of modules not only in the factory but also before shipping, before installation and after installation during their service life, to identify defective or isolated cells or any other features of modules that are related to unwanted changes in power-generation performance. It would be especially desirable to be able to determine the root cause of any identified problems.

The best-known method for inspecting modules is current-and-voltage (I-V) testing, which measures the current (I) and voltage (V) characteristics of a particular module under simulated or actual solar illumination conditions, giving a detailed description of its solar energy conversion ability and efficiency. Knowing the I-V characteristics of a module, especially its maximum power point (MPP), is critical in determining its expected output performance and solar efficiency, and hence its value. All modules are tested for I-V performance as a routine part of their manufacture.

Other common inspection technologies for modules include visual inspection with cameras under UV or visible illumination, thermography and electroluminescence, with the latter two described in M. Köntges ‘Reviewing the practicality and utility of electroluminescence and thermography images’, 2014 Photovoltaic Module Reliability Workshop, Golden, Colo., 25-26 Feb. 2014, pp 362-388. Thermography, which essentially looks for temperature differences within or between modules, is presently the most common technique for inspecting modules in the field, i.e. after installation. It may not necessarily have sufficient resolution to determine the cause of a fault, but defective modules can be removed for further investigation in module ‘autopsy’ labs, e.g. using I-V testing or electroluminescence imaging. Another shortcoming of thermography is that it can only identify faults that are already causing significant degradation of the electrical performance. In other words it is not suitable for identifying more subtle effects that could be used to predict module failure. For example thermography cannot detect cracks in cells that have not yet grown to impede current flow.

Another method for monitoring modules in the field is to log their real time performance using special circuitry integrated with the module or in the inverter that measures, for example, a module's power output as well as its operational current and voltage. This test measures the power production of a module throughout an extended period and can alert an operator to a fault in a module or even within a string within a module, but does not determine a cause of a fault. Similar to thermography, this method generally only finds faults that have evolved to a level where they lead to significant deviations of the electrical output from the rated module performance.

Full field electroluminescence (EL) imaging, in which the spatial distribution of band-to-band luminescence arising from radiative recombination of charge carriers injected through the contact terminals of a forward biased module is measured with a CCD camera or similar, is useful for detecting and locating a variety of defects in the individual cells, as well as inferring the presence of breaks or errors in the connections between cells. FIG. 3 shows in schematic side view a typical system 300 for acquiring full field EL images from a photovoltaic module 100, comprising a power supply 302 for injecting current into the module through contact terminals 106, an area camera 304 for detecting EL 306 emitted from the cells 102 within the module, and a memory 308 for storing the image read out from the camera. Because silicon is an extremely poor light emitter, full field EL imaging systems generally also require a light-proof enclosure 310 for excluding ambient light. Full field EL imaging systems are generally bulky because of the large working distance 312 required by the area camera 304, which is one reason why they are usually confined to module autopsy labs or factory inspection rather than in-the-field module inspection. The working distance 312 can be reduced somewhat if multiple area cameras 304 are used to capture EL emitted from different portions of a module 100, but this increases the cost of the apparatus.

Full field EL imaging is sensitive to many defects related to module failure, including cracks, shunts and breaks in the metal contact pattern of a cell, as well as carrier recombination defects such as dislocations and impurities that reduce the charge carrier lifetime and hence degrade cell performance. Virtually all defects tend to reduce EL emission and hence appear darker than the background defect-free material in EL images, so it can be difficult to distinguish between different types of defects. Image processing algorithms can be used to distinguish automatically between dark features with different intensities, positions, shapes, sizes and other properties, but the accuracy and precision of such algorithms can be compromised if there are a large number of types of features that may also be overlapping.

A general property of EL imaging is that luminescence is only generated from cell regions that can be accessed by the electrical excitation. This effect is illustrated in FIG. 4, showing an EL image of a module 100 with sixty multicrystalline silicon cells 102 acquired with an apparatus of the type shown in FIG. 3. Several of the cells appear completely dark, probably because they are externally shunted, e.g. by interconnection errors during manufacture, so that no charge carriers can be injected into them. While this sort of luminescence pattern is useful in revealing the presence of a module fault, the dark cells could contain defects such as cracks that clearly will not be detected. In another example, an entire module will appear completely dark under EL imaging if the interconnections between any two cells are completely broken. In general, the absence of luminescence from some or all cells in a module limits the amount of information available for defect detection or fault diagnosis.

Another luminescence-based technique that can be applied to inspection of cells and modules is photoluminescence (PL) imaging, which differs from EL imaging in that charge carriers are generated optically, by injection of high intensity light, rather than electrically. A PL-based module inspection technique is described in published US patent application No 2015/0155829 A1. In this technique a module under test is illuminated by the sun and imaged with an area camera while the working point of the module is electrically modulated at a selected frequency. This imposes a similar modulation on the PL emitted from the illuminated cells, enabling lock-in techniques to separate the PL signal from ambient light. It would appear that the ability of this technique to operate depends on the amount of sunlight available, and as with full field-EL imaging the apparatus is generally bulky. Furthermore because sunlight has significant intensity across a very broad spectrum, the spatial resolution of images is relatively poor even with the best available lock-in techniques. Such low-resolution images are generally not useful for isolating individual defects but rather can only identify cells with low PL emissions that will probably have low power generation.

There exists a need for improved apparatus and methods for inspecting or determining the condition of photovoltaic modules in the factory, before installation, in service and in module autopsy labs, to detect and locate reliably the occurrence of failure modes that adversely affect their performance. There also exists a need for a system for determining one or more conditions, such as features or defects, of photovoltaic modules throughout the service life of the photovoltaic modules, such as for determining if and when failure modes may have occurred or may be likely to occur.

### SUMMARY OF THE INVENTION

It is an object of the present invention to overcome or ameliorate at least one of the disadvantages of the prior art, or to provide a useful alternative. It is an object of the present invention in a preferred form to provide improved apparatus and methods for inspecting or determining the condition of photovoltaic modules in the factory, before installation, in service or in module autopsy labs. It is another object of the present invention in a preferred form to provide a system and method for determining one or more conditions, such as features or defects, of a photovoltaic module, preferably throughout the production, transport, installation and service life of the photovoltaic module.

In accordance with a first aspect of the present invention there is provided an apparatus for inspecting a photovoltaic module, said apparatus comprising: a power supply for applying electrical excitation to a photovoltaic module to generate electroluminescence from said photovoltaic module; a detector for detecting electroluminescence emitted from a first area of said photovoltaic module; a scanning mechanism for scanning said first area along said photovoltaic module whilst applying said electrical excitation; and a computing device programmed by executable instructions to receive, from said detector as said first area is scanned along said photovoltaic module, an image of electroluminescence emitted from at least a portion of said photovoltaic module.

In certain embodiments the detector comprises a line camera or a TDI camera. In other embodiments the detector comprises a contact imaging sensor.

In certain embodiments the scanning mechanism comprises a mechanism for moving the photovoltaic module. In other embodiments the scanning mechanism comprises a mechanism for moving the detector. In yet other embodiments the scanning mechanism comprises an optical element operatively associated with the detector, the optical element being adapted to move along the photovoltaic module while the detector remains stationary. Preferably, the scanning mechanism is configured such that the optical path length between the first area and the detector remains substantially constant as the first area is scanned along the photovoltaic module.

In preferred embodiments the apparatus further comprises one or more temperature sensors for monitoring the temperature of the photovoltaic module in the vicinity of the first area as the first area is being scanned along the photovoltaic module, for enabling a temperature correction to be applied to the electroluminescence signal detected by the detector.

Preferably, the apparatus further comprises a light source for illuminating a second area of the photovoltaic module with light suitable for generating photoluminescence from the photovoltaic module, such that an image of photoluminescence emitted from at least a portion of the photovoltaic module can be acquired as the second area is scanned along the photovoltaic module. In certain embodiments the light source and the detector are configured such that the image of photoluminescence can be acquired with the detector. In other embodiments the apparatus further comprises a second detector for acquiring the image of photoluminescence.

In certain embodiments the apparatus is configured to acquire I-V test data from the photovoltaic module, or to acquire an optical image of at least a portion of the photovoltaic module, or to acquire an image of thermal radiation emitted from at least a portion of the photovoltaic module as a result of the application of electrical excitation to the photovoltaic module.

In preferred embodiments the apparatus further comprises a computer for processing one or more electroluminescence images and/or photoluminescence images acquired with the apparatus, the computer being programmed to classify or distinguish between different types of features or defects, or generate one or more overlay images for highlighting one or more types of features or defects, or calculate one or more metrics of the occurrence of one or more types of features or defects, or apply a quality classification to the photovoltaic module, based on expected performance as estimated from the occurrence of various types of features or defects identified in the photovoltaic module. In certain embodiments the apparatus further comprises a computer for comparing two or more images of the photovoltaic module acquired with the apparatus, the images being selected from the group comprising electroluminescence images, photoluminescence images, optical images or thermal images.

In accordance with a second aspect of the present invention there is provided an apparatus for inspecting a photovoltaic module, said apparatus comprising: a light source for illuminating a second area of a photovoltaic module with light suitable for generating photoluminescence from said photovoltaic module; a detector for detecting photoluminescence emitted from a first area said photovoltaic module; a scanning mechanism for scanning said first and second areas along said photovoltaic module; and a computing device programmed by executable instructions to receive, from said detector as said first and second areas are scanned along said photovoltaic module, an image of photoluminescence emitted from at least a portion of said photovoltaic module.

The apparatus is preferably configured such that, in use, the first and second areas are at least partially overlapping.

In certain embodiments the detector comprises a line camera or a TDI camera. In other embodiments the detector comprises a contact imaging sensor.

In certain embodiments the scanning mechanism comprises a mechanism for moving the photovoltaic module. In other embodiments the scanning mechanism comprises a mechanism for moving the detector and/or the light source. In yet other embodiments the scanning mechanism comprises an optical element operatively associated with the detector, the optical element being adapted to move along the photovoltaic module while the detector remains stationary. Preferably, the scanning mechanism is configured such that the optical path length between the first area and the detector remains substantially constant as the first and second areas are scanned along the photovoltaic module.

In preferred embodiments the apparatus is configured to acquire an image of electroluminescence emitted from at least a portion of the photovoltaic module as a result of the application of electrical excitation to the photovoltaic module, or to acquire I-V test data from the photovoltaic module, or to acquire an optical image of at least a portion of the photovoltaic module, or to acquire an image of thermal radiation emitted from at least a portion of the photovoltaic module as a result of the application of electrical excitation to the photovoltaic module.

Preferably, the apparatus further comprises a computer for processing one or more photoluminescence images and/or electroluminescence images acquired with the apparatus, the computer being programmed to classify or distinguish between different types of features or defects, or generate one or more overlay images for highlighting one or more types of features or defects, or calculate one or more metrics of the occurrence of one or more types of features or defects, or apply a quality classification to the photovoltaic module, based on expected performance as estimated from the occurrence of various types of features or defects identified in the photovoltaic module. In certain embodiments the apparatus further comprises a computer for comparing two or more images of the photovoltaic module acquired with the apparatus, the images being selected from the group comprising electroluminescence images, photoluminescence images, optical images or thermal images.

In accordance with a third aspect of the present invention there is provided a method for inspecting a photovoltaic module, said method comprising the steps of: applying electrical excitation to said photovoltaic module to generate electroluminescence from said photovoltaic module; detecting, with a detector, electroluminescence emitted from a first area of said photovoltaic module; scanning said first area along said photovoltaic module whilst applying said electrical excitation; and receiving, from said detector as said first area is scanned along said photovoltaic module, an image of electroluminescence emitted from at least a portion of said photovoltaic module.

In certain embodiments the step of scanning the first area comprises moving the photovoltaic module. In other embodiments the step of scanning the first area comprises moving the detector. In yet other embodiments the step of scanning the first area comprises moving an optical element operatively associated with the detector while the detector remains stationary. Preferably, the optical path length between the first area and the detector remains substantially constant as the first area is scanned along the photovoltaic module.

In preferred embodiments the method further comprises the steps of: monitoring the temperature of the photovoltaic module in the vicinity of the first area as the first area is being scanned along the photovoltaic module; and applying a temperature correction to the electroluminescence signal detected by the detector.

Preferably, the method further comprises the steps of: illuminating a second area of the photovoltaic module with light suitable for generating photoluminescence from the photovoltaic module; and acquiring an image of photoluminescence emitted from at least a portion of the photovoltaic module as the second area is scanned along the photovoltaic module.

In certain embodiments the method further comprises the step of acquiring I-V test data from the photovoltaic module, or the step of acquiring an optical image of at least a portion of the photovoltaic module, or the step of acquiring an image of thermal radiation emitted from at least a portion of the photovoltaic module as a result of the application of electrical excitation to the module.

In preferred embodiments the method further comprises the step of processing one or more electroluminescence images and/or photoluminescence images acquired from the photovoltaic module, to classify or distinguish between different types of features or defects, or generate one or more overlay images for highlighting one or more types of features or defects, or calculate one or more metrics of the occurrence of one or more types of features or defects, or apply a quality classification to the photovoltaic module, based on expected performance as estimated from the occurrence of various types of features or defects identified in the photovoltaic module. In certain embodiments the method further comprises the step of comparing two or more images acquired from the photovoltaic module, the images being selected from the group comprising electroluminescence images, photoluminescence images, optical images or thermal images.

In accordance with a fourth aspect of the present invention there is provided a method for inspecting a photovoltaic module, said method comprising the steps of: illuminating a second area of said photovoltaic module with light suitable for generating photoluminescence from said photovoltaic module; detecting, with a detector, photoluminescence emitted from a first area of said photovoltaic module; scanning said first and second areas along said photovoltaic module; and receiving, from said detector as said first and second areas are scanned along said photovoltaic module, an image of photoluminescence emitted from at least a portion of said photovoltaic module.

Preferably, the first and second areas are at least partially overlapping.

In certain embodiments the step of scanning the first and second areas comprises moving the photovoltaic module. In other embodiments the step of scanning the first and second areas comprises moving the detector and/or the light source. In yet other embodiments the step of scanning the first and second areas comprises moving an optical element operatively associated with the detector while the detector remains stationary. Preferably, the optical path length between the first area and the detector remains substantially constant as the first and second areas are scanned along the photovoltaic module.

In certain embodiments the method further comprises the step of acquiring an image of electroluminescence emitted from at least a portion of the photovoltaic module as a result of the application of electrical excitation to the photovoltaic module, or the step of acquiring I-V test data from the photovoltaic module, or the step of acquiring an optical image of at least a portion of the photovoltaic module, or the step of acquiring an image of thermal radiation emitted from at least a portion of the photovoltaic module as a result of the application of electrical excitation to the photovoltaic module.

Preferably, the method further comprises the step of processing one or more photoluminescence images and/or electroluminescence images acquired from the photovoltaic module, to classify or distinguish between different types of features or defects, or generate one or more overlay images for highlighting one or more types of features or defects, or calculate one or more metrics of the occurrence of one or more types of features or defects, or apply a quality classification to the photovoltaic module, based on expected performance as estimated from the occurrence of various types of features or defects identified in the photovoltaic module. In certain embodiments the method further comprises the step of comparing two or more images acquired from the photovoltaic module, the images being selected from the group comprising electroluminescence images, photoluminescence images, optical images or thermal images.

In accordance with a fifth aspect of the present invention there is provided a system able to determine a condition of a photovoltaic module over time, the system comprising: one or more processors; and a memory storing computer-executable program code including instructions which, when executed by the one or more processors, configure the one or more processors to: receive module data generated by an inspection apparatus at a first point in time, wherein the inspection apparatus is configured for generating the module data for the photovoltaic module; receive one or more items of metadata associated with the module data, the one or more items of metadata including information about at least one of the module data or the photovoltaic module; store the module data and the one or more items of metadata at a network accessible storage; and determine a condition of the photovoltaic module, based at least partially on the module data and the one or more items of metadata.

The module data preferably comprises one or more of electroluminescence images, photoluminescence images, optical images, thermal images, or I-V test data.

In preferred embodiments the inspection apparatus comprises: a detector for detecting at least one of photoluminescence emitted from the photovoltaic module or electroluminescence emitted from the photovoltaic module; a scanning mechanism for scanning an area of the photovoltaic module during the detecting; and a computing device programed by executable instructions to receive, from the detector, as the module data, at least one of a photoluminescence image or an electroluminescence image of at least a portion of the photovoltaic module.

Preferably, the one or more processors are further configured to: receive additional module data generated at a second point in time by the inspection apparatus or a different inspection apparatus; and determine the condition of the photovoltaic module at the second point in time based at least partially on comparing the module data from the first point in time with the additional module data.

In preferred embodiments the one or more processors are further configured to determine the condition of the photovoltaic module by comparing the module data with prior module data generated for the photovoltaic module at an earlier time. Preferably, the one or more processors are further configured to determine, based on the condition, at least one of: a grade for the photovoltaic module; whether the photovoltaic module has a fault; whether the photovoltaic module is likely to develop a fault; or a cause of a fault in the photovoltaic module.

Preferably, the one or more processors are further configured to send, based on the condition, a communication to a computing device of at least one entity associated with manufacture, transport, installation, operation or examination of the photovoltaic module, the communication indicating the determined condition. In preferred embodiments the one or more processors are further configured to send, to a computing device of an interested party, at least one of: the module data, prior module data, or analysis data determined with respect to the photovoltaic module, or aggregated module data received for a plurality of photovoltaic modules.

In accordance with a sixth aspect of the present invention there is provided a method able to determine a condition of a photovoltaic module over time, the method comprising: receiving, by one or more processors, module data generated by an inspection apparatus at a first point in time, wherein the inspection apparatus is configured for generating the module data for the photovoltaic module; receiving, by one or more processors, one or more items of metadata associated with the module data, the one or more items of metadata including information about at least one of the module data or the photovoltaic module; storing, by one or more processors, the module data and the one or more items of metadata at a network accessible storage; and determining, by one or more processors, a condition of the photovoltaic module, based at least partially on the module data and the one or more items of metadata.

The module data preferably comprises one or more of electroluminescence images, photoluminescence images, optical images, thermal images, or I-V test data.

In preferred embodiments the inspection apparatus comprises: a detector for detecting at least one of photoluminescence emitted from the photovoltaic module or electroluminescence emitted from the photovoltaic module; a scanning mechanism for scanning an area of the photovoltaic module during the detecting; and a computing device programed by executable instructions to receive, from the detector, as the module data, at least one of a photoluminescence image or an electroluminescence image of at least a portion of the photovoltaic module.

Preferably, the method further comprises the steps of: receiving additional module data generated at a second point in time by the inspection apparatus or a different inspection apparatus; and determining the condition of the photovoltaic module at the second point in time based at least partially on comparing the module data from the first point in time with the additional module data.

Preferably, determining the condition of the photovoltaic module comprises comparing the module data with prior module data generated for the photovoltaic module at an earlier time. In preferred embodiments the method further comprises the step of determining, based on the condition, at least one of: a grade for the photovoltaic module; whether the photovoltaic module has a fault; whether the photovoltaic module is likely to develop a fault; or a cause of a fault in the photovoltaic module.

In certain embodiments the method further comprises the step of sending, based on the condition, a communication to a computing device of at least one entity associated with manufacture, transport, installation, operation or examination of the photovoltaic module, the communication indicating the determined condition. In certain embodiments the method further comprises the step of sending, to a computing device of an interested party, at least one of: the module data, prior module data, or analysis data determined with respect to the photovoltaic module, or aggregated module data received for a plurality of photovoltaic modules.

### BRIEF DESCRIPTION OF THE DRAWINGS

Benefits and advantages of the present invention will become apparent to those skilled in the art to which this invention relates from the subsequent description of exemplary embodiments and the appended claims, taken in conjunction with the accompanying drawings. In the drawings, the use of the same reference numbers in different figures indicates similar or identical items or features.

FIG. 1 shows in schematic plan view a silicon cell-based module.

FIG. 2 shows in schematic plan view a thin film module.

FIG. 3 illustrates in schematic side view a conventional apparatus for acquiring EL images of a module.

FIG. 4 shows an EL image of a silicon cell-based module acquired with an apparatus of the type shown in FIG. 3.

FIGS. 5A, 5B and 5C respectively show a schematic plan view, a schematic side view and a 3D rendered image of an apparatus for inspecting a module, according to an embodiment of the present invention.

FIG. 5D shows in schematic side view a variation of the apparatus shown in FIGS. 5A to 5C.

FIGS. 6A and 6B show in schematic plan and side views an apparatus for inspecting a module, according to another embodiment of the present invention.

FIG. 6C shows in schematic side view a compact PL line-scanning head.

FIG. 7A shows in schematic side view an apparatus for inspecting a module, according to another embodiment of the present invention.

FIG. 7B shows in schematic side view a variation of the apparatus shown in FIG. 7A.

FIG. 8A shows in schematic side view an apparatus for inspecting a module, according to another embodiment of the present invention.

FIG. 8B shows in schematic side view a variation of the apparatus shown in FIG. 8A.

FIG. 9 shows in schematic side view an apparatus for inspecting a module, according to yet another embodiment of the present invention.

FIG. 10A shows a line-scanning PL image of a module containing multicrystalline silicon cells.

FIG. 10B shows an image of a single cell extracted from the image of FIG. 10A.

FIG. 11A shows an image of a multicrystalline silicon cell extracted from a line-scanning EL image of a complete module.

FIG. 11B shows an image of the same cell as in FIG. 11A, extracted from a line-scanning PL image of the complete module.

FIGS. 11C and 11D respectively show line-scanning EL and line-scanning PL images of the corner regions of four silicon cells in a module.

FIG. 12 illustrates a high-level example of a system for determining conditions of photovoltaic modules, such as throughout their useful life.

FIG. 13 illustrates a cloud-based Software as a Service (SaaS) model for operation of the system of FIG. 12.

FIG. 14 illustrates an example physical and logical architecture of the system of FIG. 12 according to some implementations.

FIG. 15 is a flow diagram illustrating an example process for determining conditions of modules over time according to some implementations.

FIGS. 16, 17, 18 and 19 are flow diagrams illustrating example processes for generating module data according to some implementations.

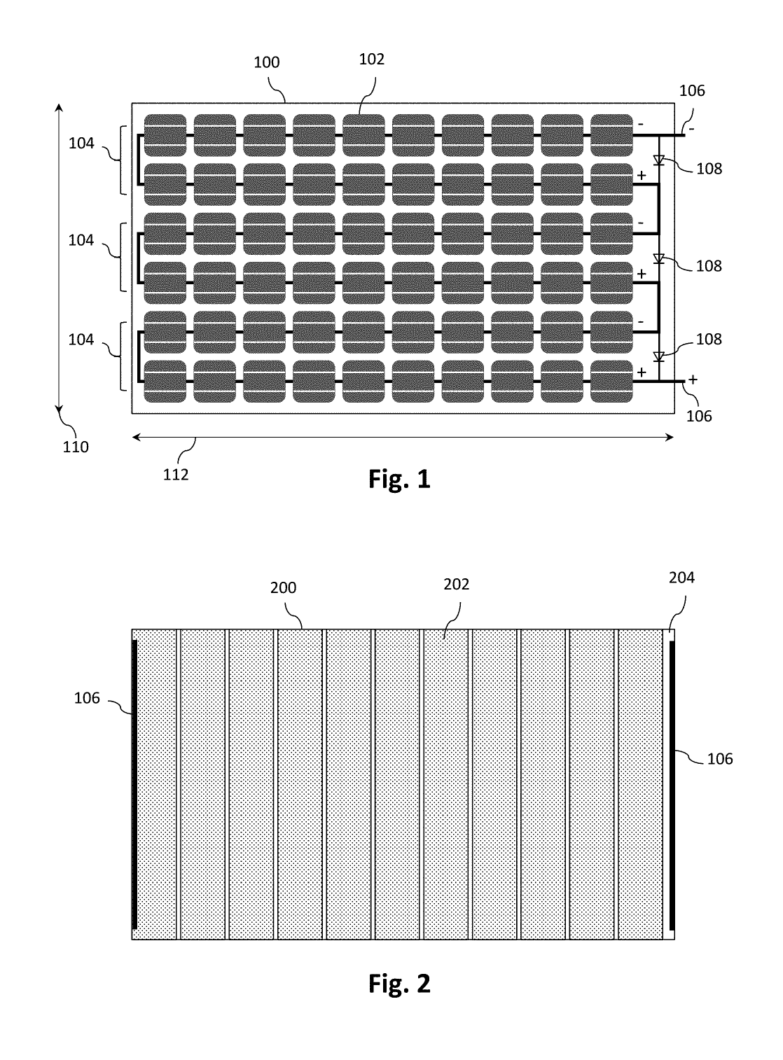

### DETAILED DESCRIPTION

Preferred embodiments of the invention will now be described, by way of example only, with reference to the accompanying drawings.

Luminescence Imaging Apparatus for Module Inspection

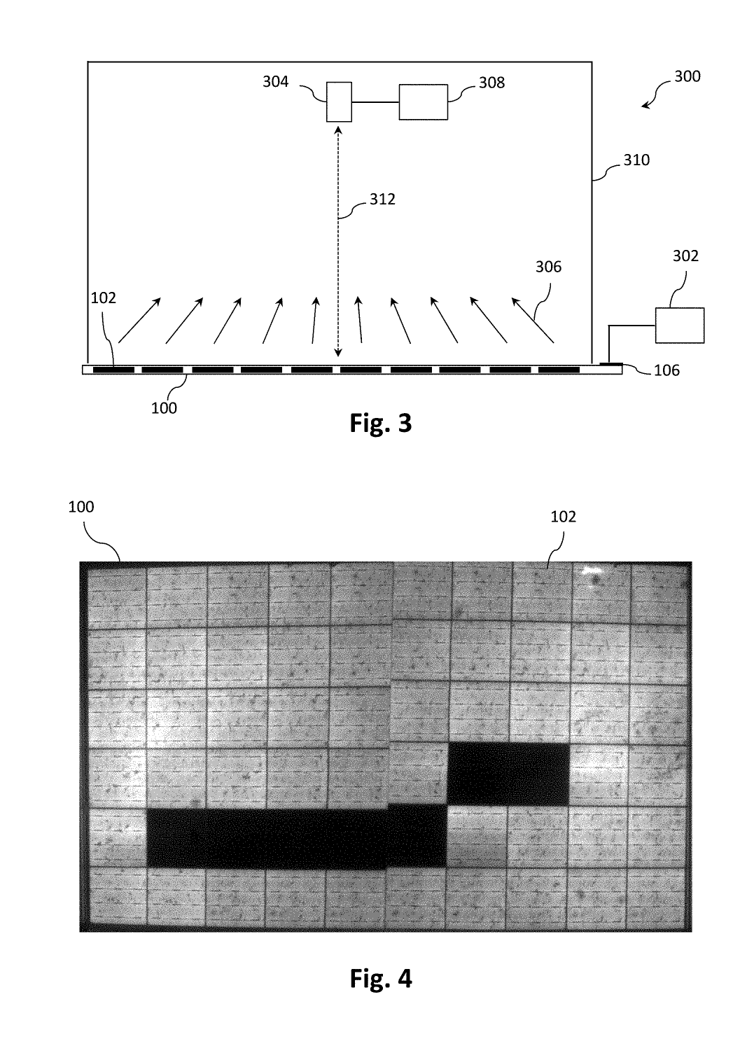

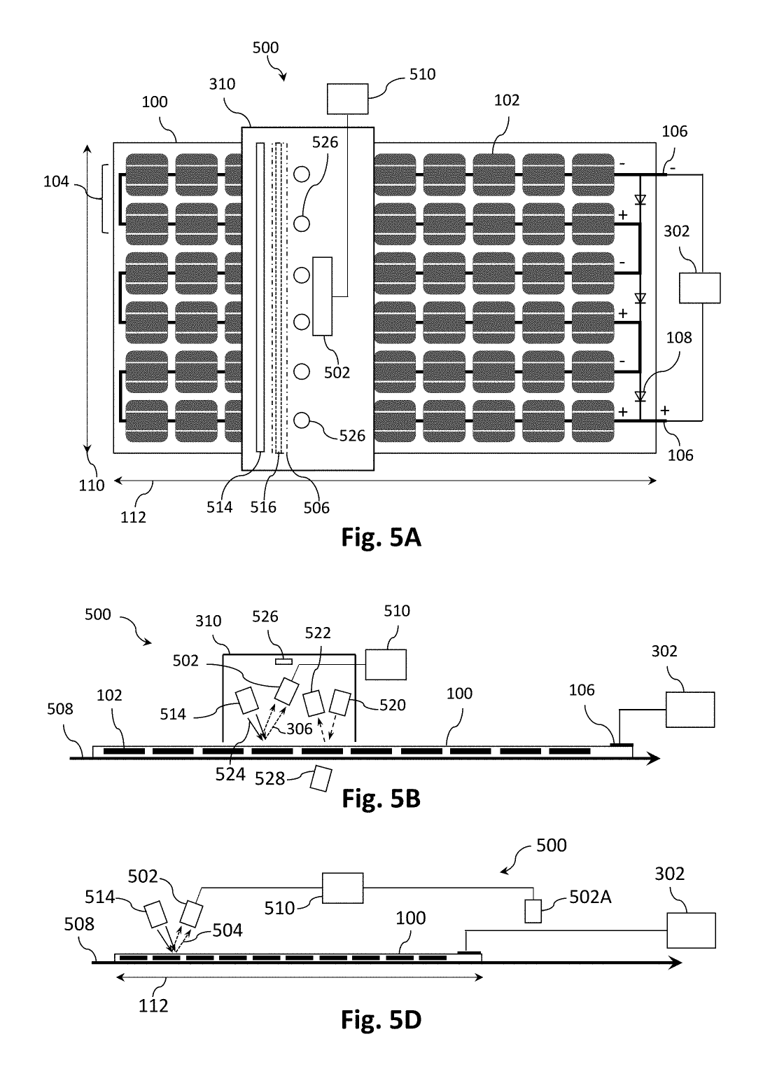

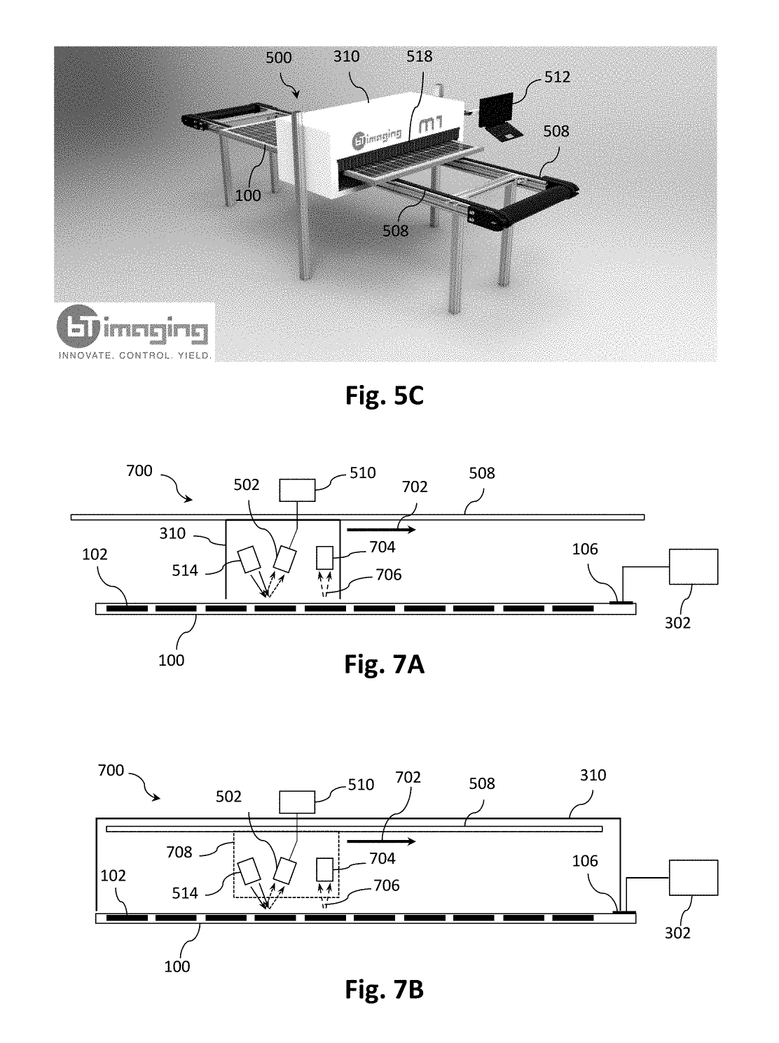

FIGS. 5A and 5B show in schematic plan and side views an apparatus 500 according to an embodiment of the present invention, for inspecting or determining the condition of a module 100 comprising a two-dimensional array of sixty silicon cells 102. A 3-D rendered image of the apparatus 500 is shown in FIG. 5C. The apparatus 500 comprises: a power supply 302 for applying electrical excitation to the module 100 via the contacts 106 to generate electroluminescence 306 from the module; a detector 502 in the form of a line or time delay integration (TDI) camera for detecting EL emitted from a first area 506 of the module; a scanning mechanism 508, such as a conveyer, rollers or air bearings, for moving the module 100 such that the first area 506 is scanned along the module; and a suitably programmed computing device 510 for reading out the camera 502 line by line in synchronisation with the scanning to obtain an image of EL emitted from at least a portion of the module. Preferably the first area 506 extends across the width 110 of the module as shown, and is scanned along the full length 112 of the module, so that the entire front surface of the module 100 is imaged. Generally, the luminescence 306 generated by the electrical excitation will primarily be band-to-band EL from the cells 102, but the possibility of generating EL from other components of a module should not be excluded. Suitable cameras for detecting band-to-band luminescence from silicon cells include silicon and InGaAs cameras. FIG. 5C also shows a terminal 512 for operator control of the apparatus 500 or for presentation of acquired images to an operator. It will be appreciated that the line-scanning EL imaging apparatus 500 depicted in FIGS. 5A to 5C may be much more compact than the area-imaging EL apparatus 300 of the prior art, as shown in FIG. 3. Although it is preferred for the generated EL 306 to be detected with a multi-pixel detector such as a line or TDI camera 502 as shown, it could alternatively be detected with a single element detector configured to move back and forth in the direction perpendicular to the direction in which the module 100 is moved.

Standard band-to-band EL can be generated from the silicon cells 102 of a module 100 by applying a relatively modest forward bias to the terminals 106, typically slightly above the open circuit voltage (Voc). For example a forward bias of around 40 V to 50 V is generally adequate for generating EL from a module with sixty silicon cells 102 each having Voc ˜0.63 V. There is also the possibility of applying a reverse bias to a module, since it is known that at large reverse bias silicon cells display breakdown behaviour which manifests as luminescence from the active cell area, potentially providing additional information on the module. However the voltages required are significantly higher than for forward biased EL, typically at least 5 to 10 V and up to more than 15 V per cell i.e. several hundred to more than 1000 V for a sixty cell module, which may raise safety concerns. This, together with the possibility of large reverse biases actually causing damage to the cells, may confine reverse bias EL to use in module autopsy labs unless the modules being inspected contain far fewer cells. To apply a sufficiently large reverse bias for generating breakdown behaviour, it will generally be necessary to disconnect or otherwise disable any by-pass diodes 108 of the subject module 100. This should be possible since by-pass diodes are usually located in a junction box, but further suggests that testing based on reverse bias EL imaging would be reserved for special cases such as module autopsy rather than for mass testing of modules.

In preferred embodiments the apparatus further comprises a light source 514 for illuminating a second area 516 of the module 100 with light suitable for generating PL from the cells 102, and possibly also from other components of the module such as the backsheet polymer. For silicon cells the light source 514 may for example comprise a laser diode array or LED array emitting light in the red or near IR region, e.g. in the range of 600 nm to 980 nm. The light source 514 and camera 502 are configured such that the camera acquires an image of PL emitted from at least a portion of the module 100 as the second, illuminated, area 516 and first, imaged, area 506 are scanned along the module by the scanning mechanism 508. Preferably the second area 516 extends across the width 110 of the module as shown, and is scanned along the full length 112 of the module, so that the entire front surface of the module 100 is imaged. The light source and camera are preferably configured such that, in use, the first and second areas 506, 516 are at least partially overlapping as shown in FIG. 5A, although this is not essential if a sufficient fraction of the photo-generated charge carriers are able to migrate out of the illuminated area 516, as discussed further below. Additional optics may also be included in the apparatus 500, such as a rod lens for focussing light from the light source 514 onto the second area 516, a short-pass filter in front of the light source 514 to prevent long wavelength tail radiation from reaching the camera 502 and a long-pass filter in front of the camera 502 to block stray excitation light. One or more interchangeable filters may be provided in front of the light source 514 and/or the camera 502 for selective excitation and/or detection of PL from the base material of the cells 102 on the one hand, or from some other material in the module, such as the backsheet polymer, on the other hand. Alternatively, the apparatus 500 may contain additional light sources or detectors with different excitation or detection bands for excitation or detection of PL from various components of a module.

In certain embodiments a single camera 502 is used to detect the generated PL or EL, as shown in FIGS. 5A and 5B, in which case a module 100 could be passed through the apparatus 500 twice, e.g. forwards then backwards, for sequential acquisition of PL and EL images. A module could also be passed through the apparatus more than once to acquire two or more PL images, e.g. where the PL is generated using different illumination intensities, illumination wavelengths or detection wavelengths, or two or more EL images, e.g. with different applied voltages. FIG. 5D shows in schematic side view a variation in which the apparatus 500 contains a first camera 502A for detecting EL generated by the power supply 302, and a second camera 502 for detecting PL 504 generated by the light source 514. Both cameras could be read out by the same computing device 510 as shown, or by separate computing devices. Having two cameras enables acquisition of separate PL and EL images without having to pass the module through the apparatus twice or reverse the direction of the scanning mechanism 508. Preferably the two cameras 502, 502A are separated in the scanning direction by a distance equal to or greater than the module length 112, or the module width if the scanning is parallel to that dimension, so that optical and electrical excitation can each be applied in isolation. For example the power supply 302 would only be activated once the module 100 has passed through the illumination zone of the light source 514.

In preferred embodiments the camera 502 and the light source 514 are mounted within a substantially light-proof enclosure 310 as shown in FIGS. 5A and 5B, to keep ambient light out of the camera or to contain the excitation light 524. As shown in FIG. 5C, in certain embodiments the bottom edges of the enclosure 310 have soft brushes 518 or similar, e.g. dark cloth, for improving the light seal. In the variation shown in FIG. 5D a single enclosure could be provided covering both cameras 502 and 502A, or separate enclosures could be provided for each camera.

The electrical excitation from the power supply 302 used to generate electroluminescence will tend to heat the cells 102, which can influence their luminescence efficiency. Consequently, when acquiring a line-scanning EL image of a module 100 a temperature gradient effect could be imposed on the image if EL collected later in the scan has been generated from cells at a higher temperature. Such an artefact can be ameliorated by monitoring the temperature of the module 100 in the vicinity of the first area 506 during the scan with one or more temperature sensors 526 such as infrared thermometers spaced apart within the enclosure 310. The computing device 510 or another computing device could then apply a temperature correction to the electroluminescence signal detected by the camera 502, e.g. using a known luminescence temperature coefficient for the cells 102. This temperature gradient effect is unlikely to occur in area imaging EL imaging systems, such as that shown in FIG. 3, where the camera 304 collects EL from all parts of a module 100 simultaneously. It is also less likely to occur when acquiring a line-scanning PL image of a module, because any local heating from the light source 514 should apply equally to each part of the module as it is being imaged.

Optionally, as shown in FIG. 5B the apparatus 500 may include a vision system comprising a light source 520 such as a linear array of white light LEDs and a suitable line or TDI camera 522 for acquiring an optical (i.e. reflection) image of at least a portion of the module 100 as it is scanned through the apparatus. The camera 522 may be read out by the same computing device 510 or a different computing device. As explained in more detail below, the optical images acquired by this vision system can provide further information on defects or other features in a module 100. In other embodiments the light source 514 and camera 502 used for PL imaging can be adapted to acquire optical images, e.g. by reducing the intensity with a neutral density filter and removing any cut-off or band-pass filters that would otherwise separate the illumination and detection bands. In another variation, the apparatus 500 may include a near IR transmission vision system having a suitable light source 520 and line or TDI camera 528 on opposite sides of the module 100. Such a transmission vision system could be used for example for micro-crack detection in modules containing bifacial cells and having glass on both sides.

It is possible for luminescence to be generated with a combination of optical and electrical excitation. For example the power supply 302 may be operated to inject current into the module 100 while the light source 514 is illuminating the module. Broadly speaking, the injection or extraction of current encourages the movement of charge carriers during luminescence image acquisition, for even further discrimination between carrier lifetime defects and series resistance defects. Some potential applications of this are discussed below in the ‘Image Analysis’ section. In alternative embodiments the power supply 302 is omitted from the apparatus 500, so that luminescence is generated solely by optical excitation. In this context, and as explained in more detail in published US patent application No 2015/0168303 A1, an EL image can be simulated by configuring the apparatus 500 such that, in use, the first area 506, i.e. the ‘imaged’ stripe, and the second area 516, i.e. the ‘illuminated’ stripe, do not overlap but are instead displaced from each other. In this case luminescence is detected from photo-generated charge carriers that migrate laterally out of the ‘illuminated’ stripe 516 before recombining radiatively. Generally speaking the main contributions to this lateral migration will be majority carrier transport through the emitter layer and the base of the cells 102, as well as electrical current flow through the front and rear surface metallisation, with minority carrier diffusion through the base material also playing a small role. In certain embodiments the apparatus 500 is equipped with a mechanism for varying the extent to which the first and second areas 506, 516 overlap on the module 100. One situation where it may be advantageous to simulate an EL image via optical excitation, rather than simply applying a voltage to the module, is in an apparatus designed to acquire both EL and PL images from a module in a single pass. Since the influence of optical excitation applied to a narrow area 516 of a module is much more localised than that of electrical excitation applied to the contacts 106, two light source/camera units could be located relatively close together, resulting in a more compact apparatus and faster scanning. In contrast, in the apparatus 500 shown in FIG. 5D the ‘PL’ camera 502 and the ‘EL’ camera 502A should be separated by a distance equal to or greater than the module dimension in the scanning direction so that an EL image can be acquired without the electrical excitation contributing to the luminescence 504 captured by the ‘PL’ camera 502.

Acquired luminescence images can be stored on the computing device 510 for subsequent processing on the same or a different computing device, or displayed on a monitor 512 for interpretation by an operator. As described below, in preferred embodiments luminescence images are processed using one or more software algorithms, e.g. to highlight various types of defects or features, before being presented to an operator for interpretation, or for triggering an automatic alert, or for transfer to a database for later viewing or comparison with images acquired from the module at different times.

As shown in the plan view of FIG. 5A, luminescence generated from the module 100 is detected with a detector 502 in the form of a line or TDI camera that is considerably shorter in lateral extent than the module width 110. Line and TDI cameras with enhanced near IR response for greater sensitivity to silicon band-to-band luminescence are readily available, and TDI cameras are particularly advantageous because of the gain enhancement provided by the summing of signals from the multiple pixel rows. However this configuration also has disadvantages, such as the need for a relatively large working distance, of order tens of centimetres, and a roll-off in detected intensity from the edges of the field of view corresponding to the ‘imaged’ stripe 506. The path length of the luminescence to a line or TDI camera 502 may be considerably longer than is shown schematically in FIG. 5B, and it will be appreciated that one or more folding mirrors may be included as required to contain the optical path within an appropriately sized enclosure 310.

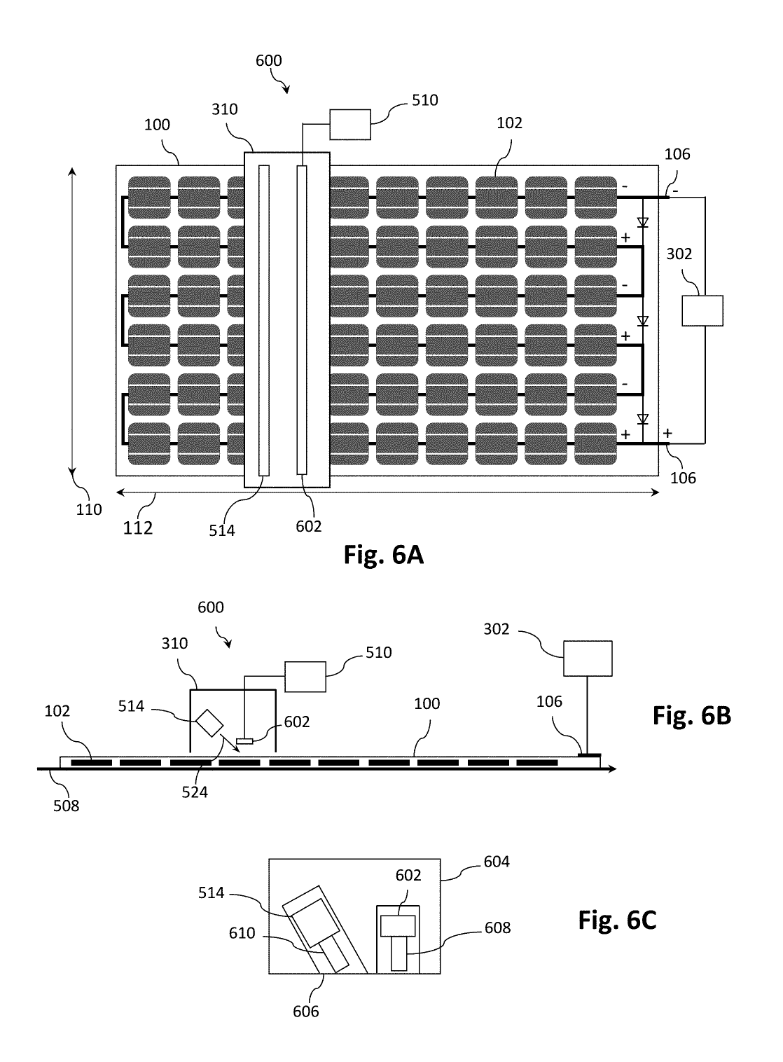

In an alternative apparatus 600 illustrated in schematic plan and side views in FIGS. 6A and 6B, the luminescence is detected using a detector in the form of a contact imaging sensor 602 for read out by a computing device 510 in synchronisation with the scanning to obtain an image of luminescence emitted from at least a portion of the module. The contact imaging sensor may for example comprise a pixel array with an integrated micro-rod lens array sufficiently long to span the full width 110 of a module 100. Contact imaging sensors of virtually unlimited length can be constructed by butting together a number of shorter CMOS sensor chips, e.g. as described in U.S. Pat. No. 8,058,602. While CMOS sensor chips are commonly used for contact imaging sensors, it is also possible to use other types of sensor, e.g. CCD sensors. In EL-only configurations, i.e. without a light source 514, a contact imaging sensor 602 can readily be placed as close as a few mm to the cover glass of a module. Additional or alternative focusing optics could be used if a somewhat larger stand-off is required, e.g. to provide better access for the illumination 524 from a light source 514 for generating PL from the module. Alternatively, as shown in schematic side view in FIG. 6C a light source 514 could be tightly integrated with a contact imaging sensor 602 to provide a highly compact PL line-scanning head 604 that could be placed as close as a few mm to the cover glass of a module. In one example a light source 514 having an output window 606 with a width in the range of 0.1 to a few mm could be located directly adjacent to, or within a few mm laterally of, the micro-rod lens array 608 of the contact imaging sensor 602. To focus its output the light source 514 could have a micro-optical array 610, which may for example have the same pitch as the micro-rod lens array 608.

Irrespective of whether it is configured for EL or PL imaging, the use of a contact imaging sensor 602 enables a compact module inspection apparatus. In another variation suitable for modules containing two-dimensional arrays of cells, the detector could be in the form of separate CMOS sensor chips provided for detecting the luminescence from each row of cells. Commercial contact imaging sensor systems are generally designed for operation in the visible spectral region, and would only be sensitive to the short wavelength end of the silicon luminescence band. This reduction in sensitivity can be offset by using arrays of rectangular sensor pixels with long axis parallel to the scan direction, preferably in combination with a micro-optical array having elements that gather light onto the rectangular sensor pixels from sample areas that have an approximately 1:1 aspect ratio (length to width), or are essentially circular. The insensitivity to long wavelength luminescence can in fact be advantageous in improving spatial resolution for reasons discussed in published PCT patent application No WO 2011/017776 A1.

Many other detection configurations are possible for collecting luminescence from close to the surface of a module, for example using optical fibre ribbons or integrated optical waveguides to guide the luminescence to a pixel array, with tapering if necessary to match the dimensions of the pixel array.

It will be noted that the apparatus 500 as depicted in FIGS. 5A to 5C is configured to span the short dimension 110 of a module 100, so that the module is conveyed in the direction parallel to its long dimension 112. There may be several reasons why this is a more convenient configuration than the alternative of spanning the long dimension, for example simpler optical design or ease of connecting the power supply 302 to the contacts 106. However there is no fundamental reason why a line-scanning luminescence imaging apparatus could not be designed to scan modules in the direction parallel to the short dimension 110, e.g. using a sufficiently long contact imaging sensor system, and in terms of speed it would in fact be advantageous to do so. All other things being equal, a 1.0 m×1.65 m module would be scanned 40% faster along its short dimension compared to its long dimension.

In the apparatus 500, 600 shown in FIGS. 5 and 6 a module 100 is moved on a scanning mechanism 508, such as a conveyer, etc., while the camera 502 or contact imaging sensor 602 and the light source 514 remain stationary. In some examples, the scanning mechanism 508 for scanning the first and second areas 506, 516 along a module comprises a mechanism such as transport belts, rollers or air bearings for moving the module 100. Such an arrangement is advantageous if the detector or light source contain delicate optics, and is generally suitable for module inspection in any situation where modules can be moved, for example during or after manufacture, before or after shipping, before installation or in a module autopsy lab.

FIG. 7A shows in schematic side view an apparatus 700 for inspecting or determining the condition of a module 100 according to another embodiment of the invention. As before the apparatus comprises a light source 514 for generating PL from the cells 102 and possibly other components of the module, a detector 502 in the form of a line or TDI camera for detecting the generated PL, and a suitably programmed computing device 510 for reading out the camera line by line in synchronisation with scanning of the illuminated and imaged areas along the module 100 to obtain an image of PL emitted from at least a portion of the module. However in this case the scanning is performed by moving the light source 514 and camera 502 as indicated by the arrow 702. In the illustrated embodiment the light source 514 and camera 502 are fixedly attached within a substantially light-proof enclosure 310 adapted to move along the module 100 on a scanning mechanism 508 comprising rails or rollers or the like. This arrangement allows the module 100 to remain stationary, suitable for inspecting modules post-installation where the module is fixed in place, e.g. on a rooftop, or if it is otherwise convenient for the module to be in a fixed position. Although it is preferred for the generated luminescence to be detected with a multi-pixel detector such as a line or TDI camera 502 as shown, it could alternatively be detected with a single element detector configured to move back and forth in the direction perpendicular to the direction in which the enclosure 310 is moved. In certain embodiments the apparatus 700 also comprises a power supply 302 for injecting current into or extracting current from the module via the contacts 106, e.g. for generating EL. In alternative embodiments, for example when inspecting installed modules, the apparatus may cooperate with existing electrical infrastructure for applying electrical excitation to the module. In yet other embodiments the light source 514 is omitted, in which case luminescence is generated solely by electrical excitation. Optionally, the apparatus 700 may include a thermal imaging line or TDI camera 704 for detecting mid-IR radiation 706 emitted from hot spots in the module 100 as a result of the application of electrical excitation to the module. If the field of view of the thermal imaging camera 704 is sufficiently close to the field of view of the camera 502, the thermal imaging camera could also perform the temperature monitoring function of the temperature sensors 526 discussed above with reference to FIG. 5B.

FIG. 7B shows in schematic side view a variation of the apparatus 700 shown in FIG. 7A, in which an assembly 708 comprising the light source 514 and the camera 502, as well as the thermal imaging camera 704 if present, is configured to move along the module 100 on a scanning mechanism 508 such as a rail inside a substantially light-proof enclosure 310.

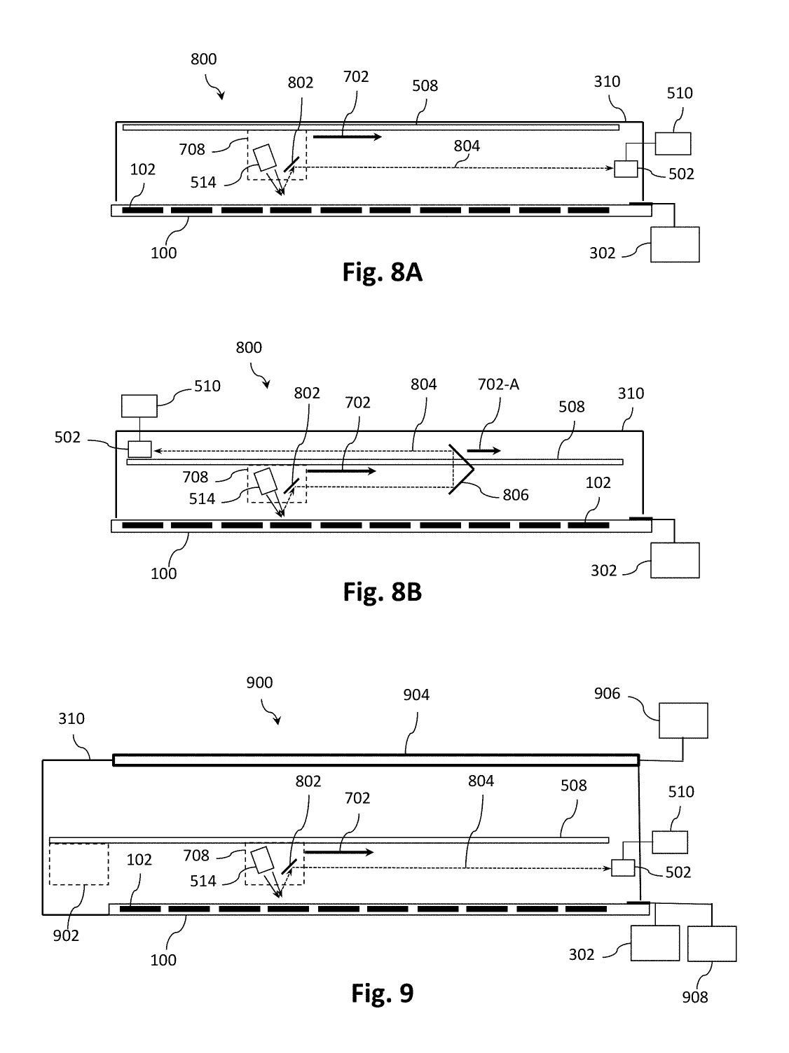

For reasons of mechanical stability it may be desirable to keep the detector fixed. FIG. 8A shows in schematic side view an apparatus 800 for inspecting or determining the condition of a module 100, according to another embodiment of the invention. In this embodiment a detector 502 in the form of a line or TDI camera is fixed within a substantially light-proof enclosure 310 placed on or around the module 100, while an assembly 708 comprising a light source 514 and an optical element 802 operatively associated with the camera 502 is adapted to move along the module 100 on a scanning mechanism 508 comprising rails or rollers or the like, as indicated by the arrow 702. The optical element 802, which may for example be an off-axis parabolic mirror, is designed to direct luminescence 804 to the camera 502 for detection and successive read-out by a suitably programmed computing device 510 in synchronisation with movement of the assembly 708 on the scanning mechanism 508. Many other optical elements suitable for directing the luminescence 804 to the camera, such as prisms and optical fibre ribbons, will occur to those skilled in the art. As before, luminescence could also be generated from the module 100 via electrical excitation from a power supply 302.

FIG. 8B shows in schematic side view a variation of the apparatus 800 shown in FIG. 8A, in which the distance travelled by the luminescence 804 to the camera 502 is kept substantially constant during scanning. As before a scanning mechanism 508 enables an assembly 708 comprising a light source 514 and an optical element 802 operatively associated with a line or TDI camera 502 to move along a module 100 while the camera 502 remains stationary, e.g. fixedly attached to a substantially light-tight enclosure 310 placed on or around the module 100. However in this embodiment the luminescence 804 generated by the light source 514 or a power supply 302 is directed to the camera 502 via a turning mirror 806 that moves on the scanning mechanism 508 at half the speed of the assembly 708 as suggested by the relative lengths of the arrows 702 and 702-A. This ensures that the distance travelled by the collected luminescence 804 to the camera 502, i.e. the optical path length between the imaged area and the camera, remains substantially constant during scanning, potentially improving the focusing onto the camera. It is noted that this is also the case with the previously described embodiments, as shown in FIGS. 5B, 5D, 6B, 7A and 7B. The detected luminescence signal is read out from the camera 502 by a suitably programmed computing device 510 in synchronisation with the movement of the assembly 708 and the turning mirror 806, to obtain an image of luminescence emitted from at least a portion of the module.

It will be appreciated that apparatus similar to that shown in FIGS. 8A and 8B, with the light source kept stationary in addition to or instead of the camera, are also possible, for example using a moving mirror to scan an illuminated area along a module under test.

FIG. 9 shows in schematic side view an apparatus 900 for inspecting or determining the condition of a module 100, according to yet another embodiment of the invention. This embodiment is similar to that shown in FIG. 8A in that luminescence 804 generated from the cells 102 and possibly other components of the module by a light source 514 or a power supply 302 is detected by a detector 502 in the form of a stationary line or TDI camera. However in this embodiment the movable assembly 708 including the light source 514 and a mirror 802 can be moved away to a resting position 902 to allow the module 100 to be exposed to a sunlight simulator 904, composed of LEDs, halogen lights or similar and controlled by a power source and controller 906. This sunlight simulator 904 can be used to simulate solar illumination of the module 100 at a range of conditions, while a power-monitoring unit 908 measures the power performance of the module including its I-V characteristics. As described in detail below, some or all of this data can be transferred to a centralised storage system and/or used locally to make decisions as to, for example, whether to proceed with installing a given module.

In each of the embodiments shown in FIGS. 5 to 9 luminescence images read out from a detector 502, and possibly optical or thermal images as well, can be stored and/or processed in a computer, which may be identified with or separate from the computing device 510 used to read out the detector, for display, automatic alerts or further analysis.

Image Analysis

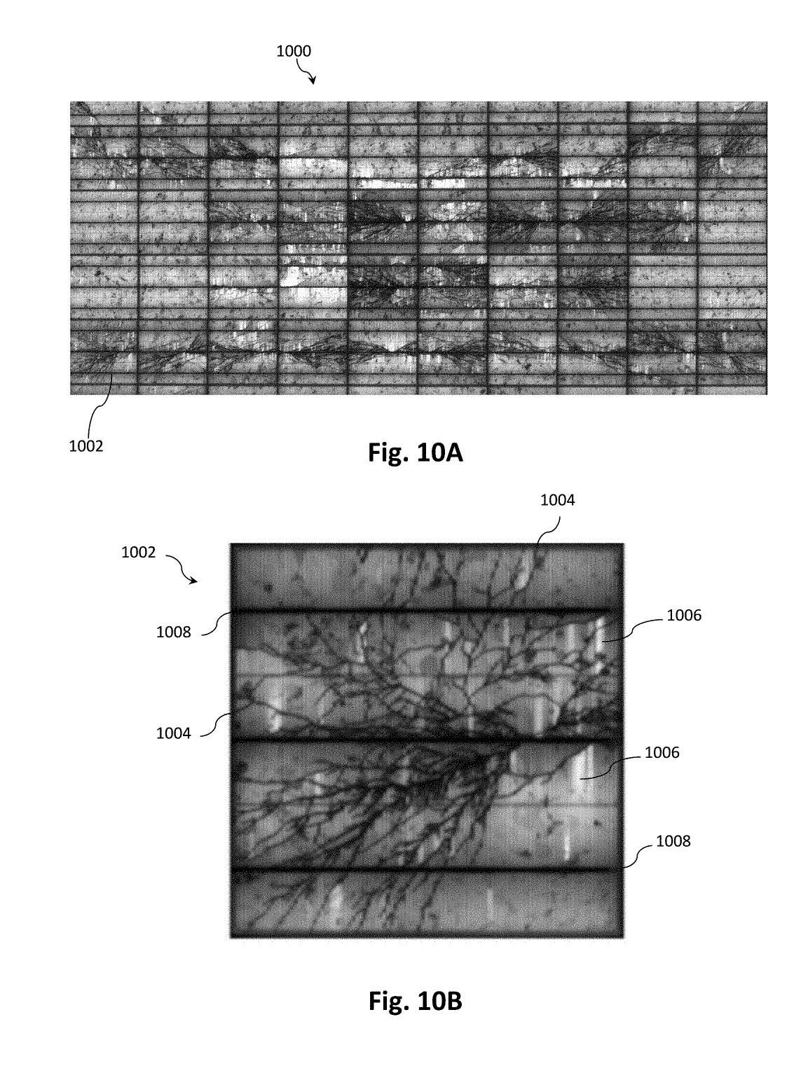

FIG. 10A shows a line-scanning PL image 1000 acquired from a substantial portion of a module having sixty multicrystalline silicon cells using an apparatus 500 of the type shown in FIGS. 5A to 5C. The image 1000 shows forty of the sixty cells in full. FIG. 10B shows the image 1002 of a single cell extracted from the image 1000. The PL was generated with near infrared illumination from an LED array and the module image 1000 captured in thirty seconds as the module was moved underneath a light source and camera assembly. The module image 1000 has approximately 70 Megapixels, representing over 1 Megapixels per cell, providing excellent spatial resolution for identifying defects or other features in individual cells as demonstrated by the single cell line-scanning PL image 1002. This image reveals an extensive network of dark lines 1004 associated with cracks, as well as a number of bright stripes 1006 extending perpendicularly to the bus bars 1008, indicative of broken metal contacts. It is a particularly useful feature of line-scanning PL images compared to EL images that defects such as cracks, dislocations or impurities causing local reduction of carrier lifetime appear relatively dark compared to the PL emission from the surrounding material, i.e. the background, whereas defects causing local increases in series resistance appear relatively bright. This ‘contrast inversion’ effect is beneficial for distinguishing different types of defects, and arises because lateral transport of photo-generated charge carriers to and along the metal conducting paths is hindered in cell areas with locally high series resistance. This increases the local concentration of carriers and hence the amount of luminescence from those areas. In areas with a high density of carrier recombination sites associated with the presence of cracks, impurities or dislocations for example, the number of carriers is reduced through local recombination so that these areas appear relatively dark.

Once one or more luminescence or other images of a module have been acquired, image processing techniques can be used to identify and quantify defects or other features appearing in the cells or other parts of the module. There are two primary tasks: defect detection and defect classification. Detection is the first step, and involves locating candidate defects and segmenting them from their surroundings. The classification step then determines the type of defect, e.g. a broken finger, crack, etc. For both of these steps it necessary to take measurements of regions of pixels that differ in intensity from the background, with these measurements referred to hereafter as ‘metrics’. Example metrics include relative intensity, size, shape, orientation, texture and position. Not all features identified by the image processing techniques will necessarily be defects that will degrade module performance, but it is important for performance-degrading defects to be identified reliably.

One of the most common metrics used for both detection and classification of defects is relative intensity, i.e. how much darker or brighter a candidate defect is compared to its surroundings. This leads to a fundamental limitation of EL-based imaging of cells, where all defects appear darker than the surrounding region. When this is the case, the ‘relative intensity’ metric does not have strong discrimination power, i.e. it is not a robust metric for differentiating one defect type from another. In contrast, and as shown in FIG. 10B, certain defect types have inverted contrast in line-scanning PL images. In particular, series resistance defects appear bright while recombination defects appear dark. In this case the ‘relative intensity’ metric has strong discrimination power and can be used to differentiate robustly between defect types.

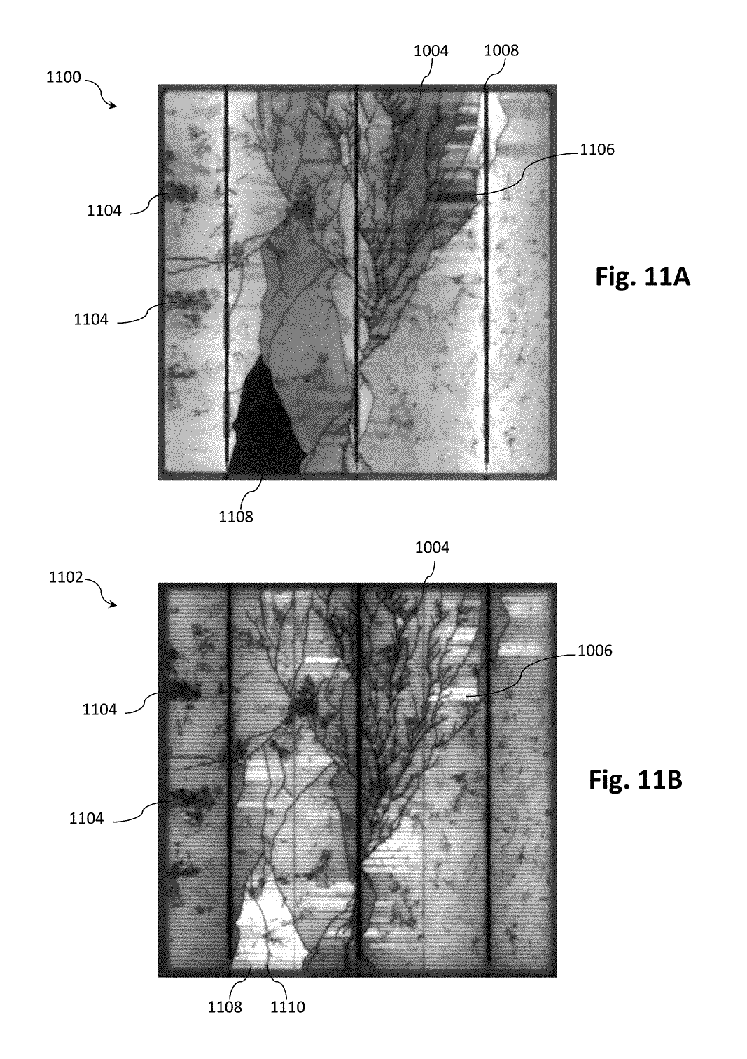

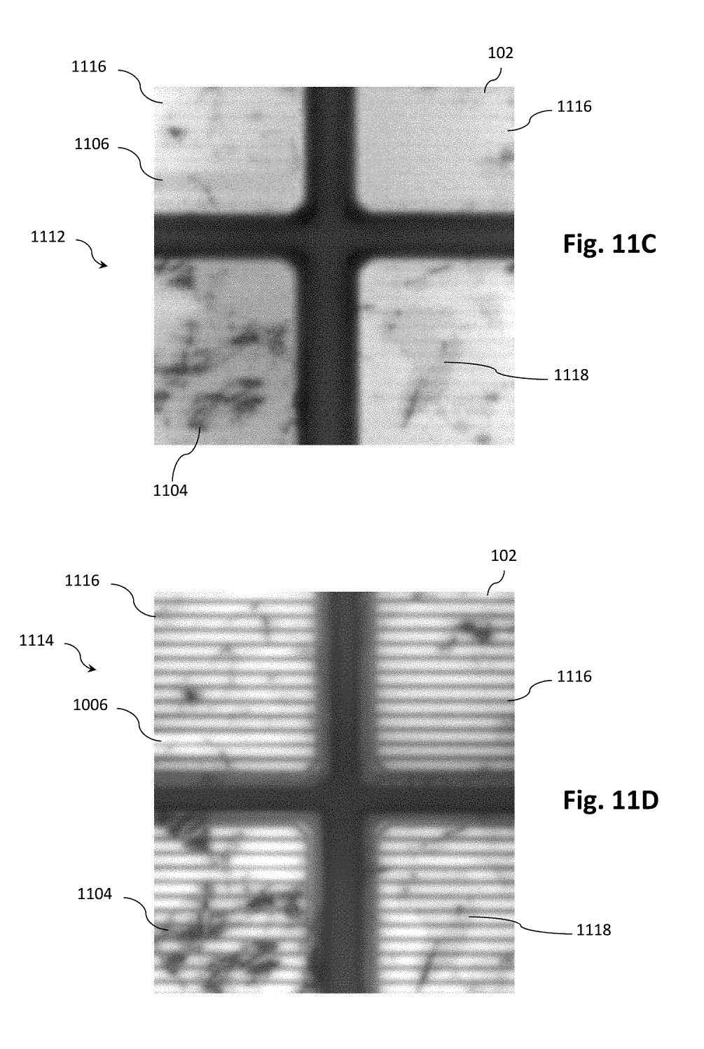

Image processing algorithms can be used to distinguish automatically between candidate defects with different relative intensities, size, shape, orientation, texture or position, among other metrics. However it will be appreciated that the accuracy and precision of such algorithms can be compromised if a sample has several types of candidate defects that can be spatially overlapping, especially if the candidate defects are all darker than the background. In this context the ‘contrast inversion’ effect in line-scanning PL images is highly beneficial in providing an additional metric that can be used to distinguish between different categories of defects, substantially improving the accuracy and precision of the image processing algorithms. The relative merits of line-scanning PL and EL imaging for cell and module inspection are further discussed with reference to the images shown in FIGS. 11A to 11D.

FIG. 11A shows a line-scanning EL image 1100 of a multicrystalline silicon cell, extracted from a line-scanning EL image of a sixty cell module acquired with an apparatus 500 such as that shown in FIGS. 5A to 5C. A forward bias of 39.5 V (equivalent to 1.045 times the open circuit voltage) was applied to the module contacts 106 as the module 100 was moved at a speed of 50 mm/s through the field of view of a silicon CCD line-scanning camera 502 with enhanced NIR response. FIG. 11B shows a line-scanning PL image 1102 of the same cell acquired with the same camera, where the PL was generated from the moving module with an illumination intensity of approximately 4 Suns from a light source 514 comprising a 1.2 m long array of near infrared LEDs focused to a 6 mm wide stripe 516 across the short side of the module.

The line-scanning EL image 1100 shows a large number of features that appear relatively dark compared to the emission from the surrounding material, including an extensive network of lines 1004 associated with cracks, dislocation clusters 1104, several dark stripes 1106 extending perpendicularly from the bus bars 1008 caused by broken metal fingers, and a large completely dark triangular region indicative of an electrically isolated cell fragment 1108. The network of cracks 1004 and the dislocation clusters 1104 appear similarly dark in the line-scanning PL image 1102, since they act as recombination centres that locally reduce the carrier lifetime. On the other hand the broken metal fingers are now revealed by bright stripes 1006, and the isolated cell fragment 1108 also appears relatively bright, because the lateral transport of photo-generated charge carriers out of these regions is partially or completely hindered. This illustrates another significant difference between EL images and line-scanning PL images. As discussed previously with reference to FIG. 4, EL can only be generated from cells or cell regions that can be accessed by the electrical excitation. In contrast it can be seen that PL can be generated across all cell regions. The ability to generate PL from within a completely isolated cell region, as demonstrated by the identification of a crack 1110 within the isolated fragment 1108, provides additional information that may be relevant for determining the cause of a cell or module failure.

A similar effect is demonstrated by comparing FIGS. 11C and 11D, which respectively show a line-scanning EL image 1112 and a line-scanning PL image 1114 of the corner regions of four multicrystalline silicon cells 102 within a module. The edges and corners of each cell are clearly visible in the line-scanning PL image 1114, whereas they are difficult to discern in the line-scanning EL image 1112 because fewer charge carriers are generated by electrical excitation in regions more distant from the metal contact fingers 1116. This effect is particularly significant for the early detection of cracks, which are often initiated at the edges of cells and are therefore more likely to be detected in a line-scanning PL image. Both images reveal a number of other features in the cells, such as several dislocation clusters 1104 in the lower left cell and some crystal grain structure 1118 in the lower right cell. The metal contact fingers 1116 are more easily discerned in the line-scanning PL image 1114. A region of locally high series resistance along one of the fingers in the upper left cell is revealed as a relatively dark stripe 1106 in the line-scanning EL image 1112 and a relatively bright stripe 1006 in the line-scanning PL image 1114, consistent with the previously noted contrast inversion.

It should be noted that although line-scanning PL images are arguably better suited than EL images for identifying different types of defects in a subject cell or module because of the contrast inversion effect, there are some module failure modes for which EL imaging may be better suited. For example an otherwise intact cell that is isolated from a module by an interconnection error may appear quite normal in a line-scanning PL image, but will appear completely dark in an EL image as shown by FIG. 4. In similar fashion cells which are partially disconnected, e.g. if one of several cell interconnects between adjacent cells is interrupted, will show a characteristic pattern with areas around certain bus bars appearing brighter than others in an EL image. Sometimes this type of pattern is sufficient to identify that specific fault mechanism. However in other cases dark patterns around bus bars can be caused by other effects, such as dark edge regions caused by high impurity concentrations in multicrystalline wafers that have been cut from edge or corner bricks. This uncertainty can be resolved by introducing a line-scanning PL image into the analysis: if an area around a bus bar appears dark in both an EL image and a line-scanning PL image it will be due to enhanced recombination, e.g. in an edge or corner wafer, whereas if the same area appears normal (i.e. without reduced intensity) in the line-scanning PL image it will be due to a cell interconnection problem. It will be appreciated that combined line-scanning PL and EL imaging apparatus such as those shown in FIGS. 5 to 9 have considerable value because of the synergy between the two imaging modes, which can yield more information than either imaging mode in isolation. Further information may also be obtained from images of EL generated with different excitation conditions such as different voltages or current injection, or images of PL generated with different illumination intensities or wavelength bands or detected in different wavelength bands, or images of luminescence generated by various combinations of optical and electrical excitation. Different combinations of luminescence images can be compared, e.g. by calculating pixel-by-pixel intensity differences or ratios, to detect or highlight certain defects or other features.

In one particular example of combined electrical and optical excitation, and with reference to FIG. 5D, injecting current into a module 100 while the light source 514 is applying illumination to the module will result in both electrical and optical excitation contributing to the luminescence 504 detected by the camera 502. The result will be a ‘biased’ line-scanning PL image that will show some characteristics of an EL image such as that shown in FIG. 11A, and some characteristics of a normal ‘unbiased’ line-scanning PL image such as that shown in FIG. 11B, with the mix depending on the relative magnitudes of the electrical and optical excitations. This may for example enable the PL imaging mode to detect cell interconnection errors that it would not otherwise be able to detect, so that a module might only need to be passed through the inspection apparatus 500 once if EL imaging is not required for any other reason. Ideally, the level of electrical excitation applied when acquiring a biased line-scanning PL image should be enough to reveal cell interconnection errors, without losing the ‘contrast inversion’ effect discussed above with reference to FIGS. 11A to 11D.

Another possibility is to extract current through the module terminals 106, e.g. with a resistor or an active load, while the light source 514 is applying illumination to the module 100. Generally, this will only yield useful information, such as an enhancement of the ‘contrast inversion’ effect, if all cells 102 in a string 104 are at least partially illuminated while one or more cells in that string are being imaged. Referring to FIG. 5A, this could be achieved if the module 100 were being scanned in the direction parallel to its short dimension 110 and the light from the light source 514 defocused such that the ‘illuminated’ stripe 516 is sufficiently wide to at least partially illuminate all cells in a string 104.

Although it is generally envisaged that the luminescence used for module inspection will be primarily generated from the cell materials, e.g. the silicon diode materials in silicon cell-based modules, an unexpected and desirable feature of the present invention is that under some circumstances it is possible to generate and detect luminescence from other materials in a module, in particular by careful selection of the light source, detector or associated optics. For example the backsheet polymeric material that is behind the cells, which may be for example be polyethylene terephthalate, polyvinylidene fluoride, polyamide or composites thereof, may be caused to emit PL. This can provide a contrasting background to the cells, and also to the metal interconnects between cells which will generally appear darker due to the lower levels of PL from metallic materials. Another example is the contact fingers on the cells, which even after firing can contain remnant organic materials from the screen printing metal pastes that may be made to luminesce, again creating useful contrast to the silicon PL. Even the metal interconnects may luminesce if the metal materials have, as is usually the case, metal oxides on their surfaces. Module components that do not luminesce can still have a detectable influence on one or more module images. In one example, oxidation-induced cloudiness of the ethylene vinyl acetate (EVA) polymer that encapsulates silicon cells within a module may be detectable from blurring of features in a luminescence or optical image, an effect that will likely be more noticeable from comparison of images acquired at different times.

One use of the unexpected contrast in the PL emitted by various components of a module is to provide an alignment test of the metal interconnects and the cells, or more specifically between the metal interconnects and the printed bus bars on the cells. Another application is to look for breaks in the metal interconnect structures. Yet another application is to probe each of the PL emitting materials for inhomogeneities in their PL emission, which can be correlated to varying material properties that may be indicative of actual or potential defects.

In certain embodiments a module inspection or condition determining apparatus is configured to acquire optical (i.e. reflection or transmission) images in addition to EL or PL images, e.g. by having an additional light source 520 and line or TDI camera 522 or 528 as shown in FIG. 5B, for obtaining further information on a module under test. For example a comparison between an optical image and a luminescence image can be useful for distinguishing carrier recombination defects such as dislocations, which will generally not be visible in an optical image, from grain boundaries which will generally be visible in both images. In another example an optical image may reveal a crack that might otherwise be hidden by a dislocation cluster. Also, a high resolution optical image may reveal defects in metal lines that can be correlated with a high series resistance region shown in a line-scanning PL image, or with the degree of darkness of the region in an EL image. Additionally, optical images may highlight defects in module components that do not luminesce, at least in response to the emission band(s) of the available light source(s). Non-luminescing module components may include packaging components such as the cover glass, the edge sealant or the polymeric encapsulant between the cover glass and the cells. Defects in the packaging components may allow the passage of oxygen and/or water to the cells or interconnects which will ultimately lead to power degrading defects such as electrical breaks or carrier recombination defects. By combining information on non-luminescing components from an optical image with information from one or more luminescence images from a number of failed modules, relationships could be developed which would allow advance warning of potential module failure even before the cells and interconnects are affected, based solely on optical images.

In yet other embodiments, a module inspection or condition determining apparatus is additionally configured to acquire images of thermal radiation emitted from at least a portion of a module, e.g. by having a thermal imaging line or TDI camera 704 as shown in FIGS. 7A and 7B, for detecting mid-IR radiation 706 emitted from hot spots in a module under test.

Module Condition Determining System

There are many situations where line-scanning imaging apparatus such as those shown in FIGS. 5 to 9 could be used to inspect modules, by acquiring images of luminescence generated by photo-excitation or electrical excitation or a combination of both, and optionally optical or thermal images or I-V test data as well. For example they could be employed in a module factory to inspect modules during production, e.g. to check strings of cells or lay-ups of cells prior to encapsulation in polymeric materials and glass, for corrective action such as replacement of cells with excessive levels of series resistance-related defects or excessive levels of cracks or other carrier recombination defects. They could also be employed in a module factory as a final test of completed modules for quality control (QC) or quality assurance (QA) purposes. Other example applications are to inspect modules after transport or before installation to check for damage caused by rough transport, or immediately after installation to check for damage caused by rough handling or improper attachment methods for example. Installed modules can also be inspected during their service life, for example as part of a periodic inspection program or after adverse events such as severe hailstorms. Finally, line-scanning imaging apparatus can be used in module autopsy laps where defective modules are examined, often as a precondition for warranty claims. Different versions of the apparatus may be designed for different applications. For example a smaller, more portable version of a ‘movable module’ apparatus of the type shown in FIGS. 5 and 6 may be designed for use outside of a factory or lab environment, e.g. to inspect modules after shipping and before installation.

It would be beneficial for warranty and determination of fault, among other purposes, to maintain a record of images acquired from a given module at these and possibly other stages, from the production line to the end of its service life.

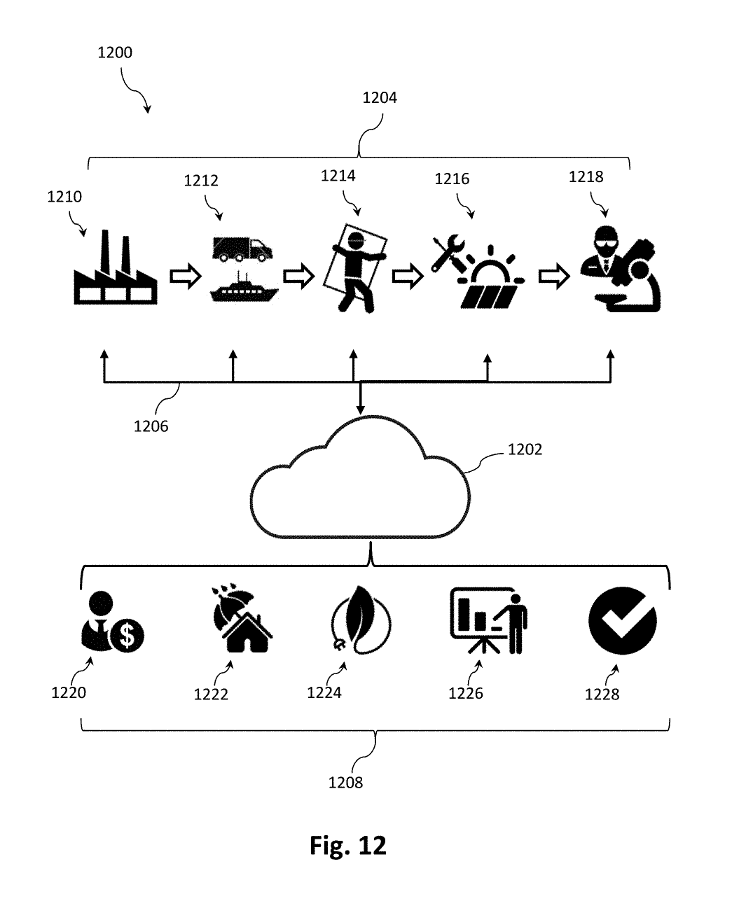

FIG. 12 illustrates a high-level example of a system 1200 for determining conditions of photovoltaic modules, such as throughout their useful life. At the centre of the system is a network accessible storage 1202 where images and other data acquired from a plurality of modules are stored. Further, ‘processed images’, i.e. images that have been processed using one or more algorithms to detect various defects and other features, may also be stored on the network accessible storage 1202. In some examples, multiple instances of the module inspection apparatus described herein may be used to determine various types of module data for a plurality of modules. The determined module data may be uploaded or otherwise sent to the network accessible storage 1202 over one or more networks 1206, such as wired or wireless data links, as discussed additionally below. Examples of such data may include photoluminescence images and/or electroluminescence images, and possibly also optical images or thermal images, and power generation and I-V test data if the module inspection apparatus is suitably equipped to monitor module power generation after installation, or if the module is inspected with an I-V test system at manufacturing or prior to installation. The data sent to the network accessible storage 1202 may be acquired by various ones of the multiple entities 1204 involved in the supply and operation of modules or the examination of failed modules, including manufacturers 1210, transporters 1212, installers 1214, module operators 1216 and module autopsy labs 1218. Data in the network accessible storage 1202 may be stored and managed by one or more servers or data centres at one or more locations.

Photovoltaic modules typically have unique or otherwise individually distinguishable identifying barcodes or numerical codes for ID purposes, which may be discernible in a luminescence or optical image, or entered manually as metadata for upload with the image(s) or other module data, or broadcast wirelessly from the inverter if the inverter is so equipped. In some examples, a plurality of metadata items associated with a module inspection event are uploaded with the images and other module data, including one or more of image acquisition apparatus ID, operator ID, time and place of image acquisition, imaging mode (e.g. EL, PL, optical or thermal), environmental conditions such as temperature and relative humidity, and operator comments. Other metadata items that can be uploaded for storage at the network accessible storage 1202 may include information on the manufacturing of the module, such as the supplier of the cells, serial numbers of the cells, type of the cells and I-V test data of individual cells. The metadata may also include detailed information about materials and processes used for module assembly, e.g. supplier and types of raw materials including wafer feedstock, and cell processing equipment and conditions such as furnaces and wafer cutting equipment. Ultimately, to gain the most value from the condition determining system, the stored data may span the entire photovoltaic value chain.

The records stored in the network accessible storage 1202 could include the geo-position of modules after installation. Combining this information with weather records for specific locations would enable development of algorithms for relating defect types with weather history for example, or to assist in assessing an insurance claim.

The module data stored at the network accessible storage 1202 can be made available for access by any of the entities 1204 involved in module supply, operation and/or examination, as well as other interested parties 1208 such as solar finance entities 1220, solar insurance entities 1222, solar energy project owners 1224, solar market reporting groups 1226 and standards and quality assurance agencies 1228, for a variety of purposes. These purposes include for example: determining which entity is at fault when a module fails to deliver its warrantied power generation; allowing insurance and finance groups to mine the data to apply risk factors to various entities in the module supply chain; allowing standards or market reporting groups to mine the data to apply quality factors to various entities in the module supply chain; allowing project owners, installers, insurers or financiers to insist upon using modules with verified testing track records prior to installation; and allowing manufacturers to provide high-quality modules that are pre-qualified with QC and QA procedures based on luminescence imaging. The module data may also provide big data for value-added analysis for any supply, operation and/or examination entity 1204 or interested party 1208, e.g. for the purposes of improvements in manufacturing, potential improvements in cell designs, suitability of specific modules for different environments, the reliability or otherwise of certain module manufacturers, transporters, or installers, and end-customer marketing. Data records containing the full history of a subject module, including information on wafer and cell manufacturing in addition to module manufacturing, can assist in tracing specific module failure modes to the use of specific materials, processes, process equipment, supplier etc.

In preferred embodiments the images uploaded to the network accessible storage 1202 are processed with one or more algorithms on a computer equipped with suitable machine-readable program code, for qualitative or quantitative identification of defects of interest. For example for luminescence images an edge detection algorithm may be applied to identify localised regions of higher or lower intensity relative to the background, that are generally indicative of defects. Other algorithms may classify or distinguish between different types of defect, e.g. based on characteristic shapes, the comparison of two or more images of luminescence generated with different excitation conditions, or the comparison of a luminescence image and an optical image. Overlay images in which different types of defect are highlighted can then be generated. Other algorithms may be applied to quantify specific types of defects. In one example a crack detection algorithm can be applied to calculate one or more metrics such as the number or total length of cracks in each cell in a module under test. Other algorithms may be applied to identify broken fingers and calculate a metric such as the number of broken fingers in each cell, or to identify and enumerate electrically isolated cells or cell regions, or to calculate metrics for carrier recombination defects such as dislocations or impurity-rich cell areas. Yet another algorithm may be used, particularly at the end of module manufacture, to apply a quality classification to a module based on expected performance as estimated from the occurrence of various types of defects identified in the module. These and other image processing outcomes from a given luminescence, optical or thermal image can be stored with that image, along with any I-V test data.

In other embodiments, image processing algorithms are applied and analytical data calculated by the supply, operation and/or examination entities 1204 that acquired the images, instead of or in addition to a computing device of a service provider associated with the network accessible storage 1202. In yet other embodiments, stored images can be analysed at the request of any of the supply, operation and/or examination entities 1204 and/or the interested parties 1208.

It will be appreciated that images and data of a given module acquired at different times, e.g. before and after transport, can be compared e.g. by subtraction or by calculation of intensity ratios to highlight any new defects, to assist in determining cause and time of module failure. Additionally or alternatively, comparisons can be made between one or more metrics obtained from those images and data. ‘Difference’ or ‘ratio’ images can be particularly useful for distinguishing newly formed defects such as cracks or broken metal fingers from carrier recombination defects such as dislocations that were present in the cell material from the beginning. Image metadata can also provide useful information, e.g. to identify whether a statistically significant number of module failures are associated with specific manufacturers 1210, transporters 1212 or installers 1214.

In certain embodiments statistical data for various groupings of modules, e.g. modules from specific manufacturers 1210 or shipped by specific transporters 1212, may be calculated by a computing device of the service provider associated with the network accessible storage 1202, either routinely or on request from an interested party 1208 or a supply, operation and/or examination entity 1204. More complex comparisons of processed module data are also possible, including comparing data obtained from images or associated metrics for a selection of one or more modules with data obtained from a general population of modules, e.g. according to an ANOVA (analysis of variance) or other statistical analysis known in the art. Similar statistical analyses can be applied to individual cells. For example PL images of one or more modules can be segmented into individual cell images that are optionally corrected for distortions before a cell template is calculated by averaging or obtaining the median of the cell images. For this purpose a module image is segmented into individual cell images, which are optionally corrected for distortions before being fed into the template calculation, and the individual images are then analysed using the average median or any other method to create a cell image of a ‘normal cell’. Suspected defective cells, i.e. cells for which the PL image deviates strongly from the template according to an ANOVA analysis or similar can then be excluded, to provide an image representative of a ‘normal cell’. Individual cell images can then be compared to the ‘normal cell’ image, which enables quantifying deviations in cell performance from the expected normal performance.

Actionable decisions can be made based on one or more of the image processing and analysis outcomes. Such decisions include for example rating a module as defective, grading a module based on expected performance, determining the likely entity at fault if a module failure is detected, and/or removing the module from service e.g. by deciding not to ship or install it. In some embodiments these decisions may be made at the network accessible storage 1202, which may serve as a centralised image storage and processing service operated as a cloud service, i.e. through an IT network and a backend server/processing unit represented in FIG. 12 as a cloud 1202. Actionable decisions can then be conveyed to an appropriate operator. In other embodiments actionable decisions can be made during module production, for example to remove defective cells or strings and replace them prior to the irreversible step of encapsulating the cells in the module packaging.

Generally there will be a cost associated with storing module data, such as image data and associated metadata, and analysis data, at the network accessible storage 1202, depending among other factors on the size of the data files being stored, the required accessibility of the data and the required storage time which can be expected to be twenty or twenty five years according to the warrantied operational life of modules, or even longer. Irrespective of any data compression algorithms that may be applied, the size of an image data file for a module will generally scale with the spatial resolution, i.e. the number of pixels. Higher resolution images may provide superior defect detection outcomes but may be more expensive to store, resulting in a trade-off. If the spatial resolution offered by an imaging system exceeds requirements, pixel binning can be used to reduce the resolution and therefore the image file size. For example the counts from 2×2 groups of pixels can be combined to reduce the image file size by a factor of four. In one particular example, the Applicant has found that 2×2 pixel binning can be applied to a 70 Megapixel luminescence image of a module, such as that shown in FIG. 10A, without markedly affecting the outcomes of the image processing algorithms as compared to the original un-binned images.

In another approach for reducing data storage requirements and costs, the Applicant has developed a proprietary data format (with a related codec) that uses 10 bits per pixel. This is decoded to 16-bit before image display or processing, which involves a small computational overhead but provides significant storage savings. Image compression is lossless in terms of resolution, so that processing and/or comparison of images in a processor associated with the network accessible storage 1202 is not compromised. In one particular example the Applicant has determined that storing two images, e.g. line-scanning EL and line-scanning PL images, in uncompressed form requires approximately 100 Megabytes of storage, compared with only 25 Megabytes for the compressed images.

In yet another approach for reducing storage costs, module data can be initially stored in faster access storage until the subject module has been installed, and thereafter moved into less expensive, slower access storage.

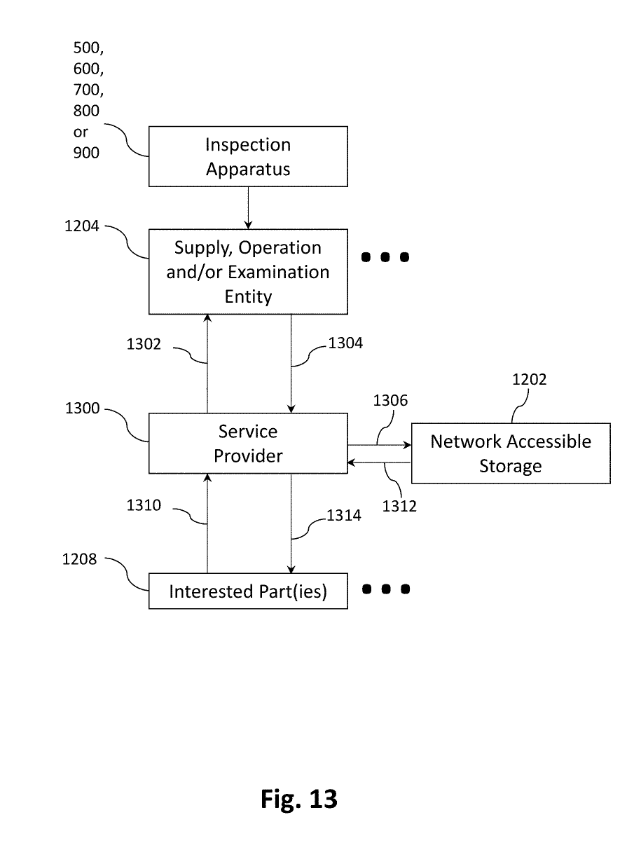

In some examples, the condition determining system 1200 shown in FIG. 12 may be operated as a network-based Software as a Service (SaaS) model. In one particular embodiment shown in FIG. 13, a service provider 1300 responsible for or otherwise associated with the module condition determining system may provide (e.g. lease, sell, etc.) as indicated at 1302, module inspection apparatus 500, 600, 700, 800 or 900 to one or more of the entities 1204 involved in the supply, operation and/or examination of modules. The entity 1204 and/or the inspection apparatus 500-900 uploads module image data 1304 to the service provider 1300 for processing, analysis, and storage 1306 at the network accessible storage 1202 or similar. The service provider 1300 may pay an operator of the network accessible storage 1202 for the data storage and may recoup the cost by charging a fee 1310 to an interested party 1208, such as a solar insurance company assessing a warranty claim, or some other interested party 1208 as enumerated above. The service provider 1300 or the interested party 1208 may retrieve 1312 and provide 1314 the requested module data and/or analysis data. In an alternative embodiment, the service provider 1300 may provide the module inspection apparatus 500-900 to a supply, operation and/or examination entity 1204 for no upfront cost, and may charge a fee to the entity 1204 for uploading or otherwise providing the module data 1304. In yet other embodiments, the service provider 1300 and a supply, operation and/or examination entity 1204 that uses the module inspection apparatus 500-900 may negotiate a higher equipment lease or sale cost in exchange for a lower fee for access to the module data. In an alternative embodiment the service provider 1300 provides module inspection equipment to a party 1204 for no upfront cost, and charges a fee for image data upload 1304 and another fee to any other party that wants to access the image data at any time in the future. Other variations of fees and charges can be considered.

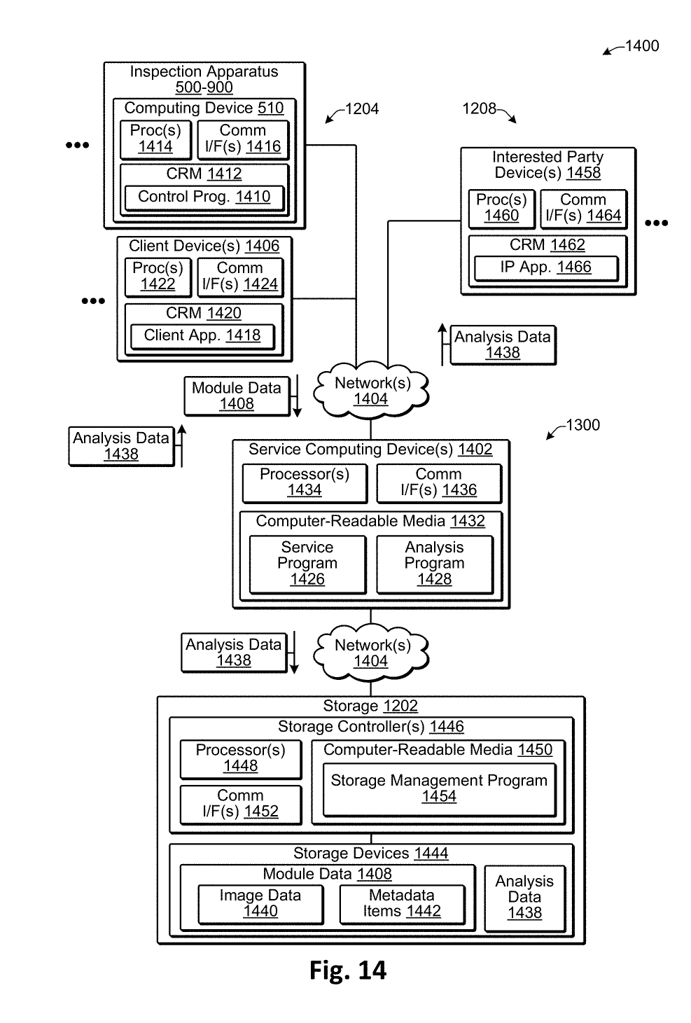

FIG. 14 illustrates an example physical and logical architecture 1400 of a system 1200 for determining conditions of photovoltaic modules (not shown in FIG. 14) according to some implementations. The architecture 1400 includes one or more service computing devices 1402 of a service provider, such as the service provider 1300 discussed above with respect to FIG. 13 or another service provider. The one or more service computing devices 1402 are able to communicate over one or more networks 1404 with the network accessible storage 1202. Further, the one or more service computing devices 1402 are able to communicate over the one or more networks 1404 with entities 1204 involved in the supply, operation and/or examination of photovoltaic modules. For example, the one or more service computing devices 1402 may communicate with client computing devices 1406 of entities 1204 involved in the supply, operation and/or examination of photovoltaic modules, and/or computing devices 510 associated with module inspection apparatus 500-900.

In some examples, the computing device 510 associated with an inspection apparatus 500-900 may be configured to send module data 1408 directly over the one or more networks 1404 to the service computing device(s) 1402, e.g. as the module data 1408 is obtained in the field. For instance, a control program 1410 may be stored or otherwise maintained in one or more computer readable media (CRM) 1412 in the computing device 510. The control program 1410 may be executed by one or more processors 1414 of the computing device 510 to obtain the module data 1408 in the field. For example, the control program 1410 may be executed to operate the camera(s), scanning mechanisms, and other components discussed above to obtain module data 1408 regarding one or more photovoltaic modules being inspected by one or more of the inspection apparatus 500-900. As mentioned above, the module data 1408 may include one or more PL and/or EL images, optical images, or other types of images, I-V test data and the like. Further, the module data 1408 may include metadata about the photovoltaic module being tested, the test being performed, the inspection apparatus performing the testing, and/or other metadata, as discussed above.

Execution of the control program 1410 may cause the processor(s) 1414 to use one or more wireless and/or wired communication interfaces 1416 to connect to the one or more networks 1404 for sending the module data 1408 to the service computing device(s) 1402. In some cases the module data 1408 may be sent in real time, e.g. as the inspection apparatus 500-900 generates the module data 1408. In other cases the module data 1408 may be sent as a batch, such as after a certain trigger point is reached, after a certain amount of data has been collected, after a certain point in time has passed, or the like. Thus, the one or more service computing devices 1402 may receive the module data 1408, store the module data 1408 at the network accessible storage 1202, and perform analysis or other operations on the module data 1408.

Additionally or alternatively, the module data 1408 may be received by the client computing device 1406 from the computing device 510 of the inspection apparatus 500-900. Subsequently, the client computing device 1406 may send the module data 1408 to the service computing device(s) 1402 for storage on the network accessible storage 1202. For example the client computing device 1406 may include a client application 1418 stored or otherwise maintained on one or more CRM 1420. The client application 1418 may be executed by one or more processors 1422 of the client computing device 1406, such as to receive the module data 1408 from the inspection apparatus 500-900 and send the module data 1408 to the service computing device(s) 1402. The client application 1418 may cause the processor(s) 1422 to use one or more wireless and/or wired communication interfaces 1424 to connect to the one or more networks 1404 for sending the module data 1408 to the service computing device(s) 1402. In some cases, the client application 1418 may be downloaded or otherwise provided to the client device 1406 by the service computing device(s) 1402. For instance the client application 1418 may be a program that specifically configures the client computing device 1406 to receive and process module data 1408 from the inspection apparatus 500-900, and to send the module data 1408 to the service computing device 1402.

In some examples one or more of the supply, operation and/or examination entities 1204 may each operate an inspection apparatus 500-900 and a client computing device 1406. For instance a module manufacturer may use an inspection apparatus 500-900 to obtain first module data about each manufactured module, and this first module data may be sent to the service computing device(s) 1402 for storage at the network accessible storage 1202. Subsequently, after a particular module has been transported to an installation location, that module may again be inspected using an inspection apparatus 500-900 to obtain second module data about that module, which may be sent to the service computing device(s) 1402 for storage at the network accessible storage 1202. Additionally, following installation that particular module may again be inspected using an inspection apparatus 500-900 to obtain third module data about that module, which may be sent to the service computing device(s) 1402 for storage at the network accessible storage 1202. Additionally, following installation that particular module may be periodically re-inspected using an inspection apparatus 500-900 to obtain additional module data about that module, which may be sent to the service computing device(s) 1402 for storage at the network accessible storage 1202. Furthermore, if that particular module is determined to have a faulty condition, it may again be inspected using an inspection apparatus 500-900 by a module autopsy lab entity to obtain still additional module data, which may be sent to the service computing device(s) 1402 for storage at the network accessible storage 1202. The module data obtained at different points in time may be compared with each other for determining when an event may have occurred that led to damage, failure or other faulty condition of the module, such as for determining a likely cause of the faulty condition of the module.

In some examples the service computing device(s) 1402 may include a service program 1426 and an analysis program 1428 stored or otherwise maintained on one or more CRM 1432. For instance the service program 1426 may be executed by one or more processors 1434 to configure the service computing device(s) 1402 to receive and process module data 1408 from an inspection apparatus 500-900 and/or the client device(s) 1406, and to send the module data 1408 to the network accessible storage 1202. The service computing device(s) 1402 may for example include one or more communication interfaces 1436 configured for communicating over the one or more networks 1404 with the inspection apparatus 500-900, the client computing devices 1406, the network accessible storage 1202 and the like.

In addition the analysis program 1428 may be executed by the one or more processors 1434 for analysing the module data 1408 to determine analysis data 1438. The analysis data 1438 may indicate conditions of particular modules and/or overall trends, causes of failure in individual or multiple modules, or the like. For example the analysis program 1428, when executed by the one or more processors 1434, may cause the processors to compare module data 1408 received for a particular module at a first point in time with module data 1408 received for that module at a second point in time to determine at least one of a quality grade for the photovoltaic module, whether the photovoltaic module has a fault, whether the photovoltaic module is likely to develop a fault, or a cause of a fault in the photovoltaic module. Additionally, the analysis data 1438 may indicate a point in the manufacturing and installation chain at which a faulty condition was first identified for determining an entity that is likely to be the cause of the faulty condition. Consequently, the analysis data 1438 may enable identification of a cause of failure or other faulty condition to enable improvement of processes for improving quality and/or reliability of modules.

The analysis data 1438 and the module data 1408, including image data 1440 and metadata 1442, may be stored on the network accessible storage 1202 on a plurality of storage devices 1444 associated with the network accessible storage 1202. The network accessible storage 1202 may provide storage capacity for the service provider 1300, as well as providing storage services for others in some examples. The network accessible storage 1202 may include storage arrays such as network attached storage (NAS) systems, storage area network (SAN) systems, or storage virtualisation systems. Further, the network accessible storage 1202 may be co-located with one or more of the service computing devices 1402, or may be remotely located or otherwise external to the service computing devices 1402.

In the illustrated example the network accessible storage 1202 includes one or more storage computing devices referred to as storage controller(s) 1446, which may include one or more servers or any other suitable computing devices, such as any of the examples discussed with respect to the service computing device(s) 1402. The storage controller(s) 1446 may each include one or more processors 1448, one or more computer-readable media 1450 and one or more communication interfaces 1452. Further, the computer-readable media 1450 of the storage controller 1446 may be used to store any number of functional components that are executable by the processor(s) 1448. In many implementations these functional components comprise instructions, modules, or programs that are executable by the processor(s) 1448 and that, when executed, specifically program the processor(s) 1448 to perform the actions attributed herein to the storage controller 1446. For example a storage management program 1454 may control or otherwise manage the storage of module data 1408 and analysis data 1438 in a plurality of storage devices 1444 coupled to the storage controller 1446.

In addition the storage devices 1444 may in some cases include one or more arrays of physical storage devices. For instance the storage controller 1446 may control one or more arrays, such as for configuring the arrays in a RAID (redundant array of independent disks) configuration or other desired storage configuration. The storage controller 1446 may provide logical units based on the physical storage devices 1444 to the service computing device(s) 1402, and may manage the data stored on the underlying physical devices 1444. The physical devices 1444 may be any type of storage device, such as hard disk drives, solid-state devices, optical devices, magnetic tape and so forth, or combinations thereof.

Additionally, the one or more service computing devices 1402 may be able to communicate over the one or more networks 1404 with computing devices 1458 of one or more interested parties 1208. The interested party computing devices 1458 include one or more processors 1460, one or more computer-readable media (CRM) 1462 and one or more communication interfaces 1464. An interested party (IP) application 1466 may be stored or otherwise maintained on the CRM 1462 and may be executed by the one or more processors 1460, e.g. for communicating with the service computing device(s) 1402 and/or receiving analysis data 1438 from the service computing device(s) 1402.

In some examples the one or more service computing devices 1402 and the storage controller(s) 1446 may include a plurality of physical servers or other types of computing devices that may be embodied in any number of ways. In the case of a server for instance, the modules, programs, other functional components, and a portion of data storage may be implemented on the servers, such as in a cluster of servers, e.g. at a server farm or data centre, a cloud-hosted computing service, and so forth, although other computer architectures may additionally or alternatively be used. Further, in some examples the client computing device(s) 1406 and/or the interested party computing device(s) 1458 may be one or more servers, or alternatively, may be personal computers, laptop computers, workstations, tablet computing devices, mobile devices, smart phones, wearable computing devices, or any other type of computing device able to send data over a network.

Each of the processor(s) 1414, 1422, 1434, 1448 and/or 1460 may be a single processing unit or a number of processing units, and may include single or multiple computing units or multiple processing cores. The processor(s) may be implemented as one or more central processing units, microprocessors, microcomputers, microcontrollers, digital signal processors, state machines, logic circuitries, and/or any devices that manipulate signals based on operational instructions. For instance the processor(s) may be one or more hardware processors and/or logic circuits of any suitable type specifically programmed or configured to execute the algorithms and processes described herein. The processor(s) may be configured to fetch and execute computer-readable instructions stored in their respective computer-readable media 1412, 1420, 1432, 1450 and/or 1462, which can program the processor(s) to perform the functions described herein.

The computer-readable media 1412, 1420, 1432, 1450 and/or 1462 may include volatile and nonvolatile memory and/or removable and non-removable media implemented in any type of technology for storage of information such as computer-readable instructions, data structures, program modules, or other data. For example the computer-readable media may include, but are not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other media that can be used to store the desired information and that can be accessed by a computing device. Depending on the configuration of the respective computing device, the computer-readable media may be a tangible non-transitory medium to the extent that, when mentioned, non-transitory computer-readable media exclude media such as energy, carrier signals, electromagnetic waves, and/or signals per se.

In some cases the computer-readable media 1412, 1420, 1432, 1450 and/or 1462 may be at the same location as the associated computing device, while in other examples the computer-readable media may be separate or partially remote from the associated computing device. Further, the computer-readable media 1412, 1420, 1432, 1450 and/or 1462 may be used to store any number of functional components that are executable by the respective associated processor(s), as discussed above. In many implementations these functional components, e.g. the control program 1410, the client application 1418, the service program 1426, the analysis program 1428, the storage management program 1454, and the interested parties application 1466, comprise instructions, modules, or programs that are executable by the respective processor(s) and that, when executed, specifically program the processor(s) to perform the actions attributed herein to the respective computing devices.

The communication interface(s) 1416, 1424, 1436, 1452 and/or 1464 may include one or more interfaces and hardware components for enabling communication with various other devices, such as over the one or more networks 1404. Thus, the communication interfaces may include, or may couple to, one or more ports that provide connection to the network(s) 1404 for communication with other computing devices. For example the communication interface(s) may enable communication through one or more of a LAN (local area network), a WAN (wide area network), the Internet, cable networks, cellular networks, wireless networks (e.g. Wi-Fi) and wired networks (e.g. Fibre Channel, fibre optic, Ethernet), direct connections, as well as close-range communications such as BLUETOOTH® and the like, as additionally enumerated elsewhere herein. In addition, the one or more networks 1404 may include wired and/or wireless communication technologies. Components used for the network(s) 1404 can depend at least in part upon the type of network, the environment selected, desired performance and the like. The protocols for communicating over the networks herein are well known and will not be discussed in detail. Further, while an example of a system architecture has been described with reference to FIG. 14, numerous other software and/or hardware configurations will be apparent to those of skill in the art having the benefit of the disclosure herein.

Operation of the module condition determining system 1200 shown in FIG. 12 is described in the following examples.

### Example 1

A manufacturer 1210 of monocrystalline silicon modules used a line-scanning EL/PL inspection apparatus for quality control testing of completed modules prior to packaging and transport. Specific modules are identifiable in line-scanning PL images by front-facing barcodes and also by numeric codes on the edge of the module frame that can be included in the metadata. Application of automatic image processing algorithms to acquired EL and PL images indicated that a specific module had no cracks, minimal series resistance issues and no interconnect issues. Consequently this module was packaged and shipped, whereas if the level of cracks for example had been above a predetermined threshold it would have been rejected and scrapped. The module was also tested for power output using a solar simulator and found to be in the category of 300 W modules. This rated power output is the basis for pricing the module.

Specific data from the luminescence imaging test and the power test were sent to the service provider 1300 of the condition determining system for storage in the network accessible storage 1202. The module data 1408 that was sent included: (i) line-scanning PL image; (ii) line-scanning EL image; (iii) I-V curve; (iv) time and date of test; (v) operator ID; (vi) factory and production line ID; (vii) module ID; (viii) crack metrics from processed EL and PL images; (ix) series resistance metrics from processed EL and PL images; (x) cell interconnect metrics from processed EL and PL images; and (xi) carrier recombination defect metrics from processed EL and PL images.

At some later time the same module was unpacked from its packaging at a commercial solar installation site. The installer 1214 used a portable version of a line-scanning EL/PL inspection apparatus to check each module prior to installation, with the objective of identifying modules that were already defective or likely to fail during the module's service period. Their motivation for doing so is related to the cost of replacing a module. The cost of replacing a single defective solar module at this site was estimated to be US 800, i.e. US 2.67 per Watt, inclusive of a US 1.66 per Watt cost for a module autopsy report on which basis a warranty claim can be made. Many manufacturer warranties require expensive autopsy tests and reports prior to any claim being made, which is aimed as a disincentive for warranty claims. Because of this cost, the project owner 1224 who had financed the installation insisted the installers 1214 spend US 1.33, i.e. US 0.0044 per Watt (inclusive of labour), to test each module with a line-scanning EL/PL inspection apparatus prior to installation. Any modules that failed the test were to be returned to the manufacturer 1210 for a refund or a replacement module. This requirement was based on the calculation that if just 0.15% of the modules failed during their 25-year service life, then identifying defective modules before installation was a lower cost option than replacing them after failure. The portable field unit for line-scanning EL- and PL-based module inspection performed the same tests as the factory version, except for I-V testing. The following module data 1408 was generated at the installation site and uploaded to the service provider 1300: (i) line-scanning PL image; (ii) line-scanning EL image; (iii) time and date of test; (iv) operator ID; (v) module ID; (vi) crack metrics from processed EL and PL images; (vii) series resistance metrics from processed EL and PL images; (viii) cell interconnect metrics from processed EL and PL images; and (ix) carrier recombination defect metrics from processed EL and PL images.

An initial test at the installation site for the ‘defectiveness’ of the subject module was based on results (vi) to (ix) of the above list. The module passed these tests, with each of the defect levels being less than the predetermined thresholds for module rejection. However before proceeding with installation, another set of data analyses was undertaken in a computing device 1402 of the service provider 1300 after upload of the module data (i) to (ix) to check for significant variations between the module data before transport and at the point of installation to check for damage that occurred during transport. Difference images were calculated by pixel-by-pixel subtraction of intensities in the ‘factory’ and ‘field’ PL images, and likewise for the two EL images. Alternatively, ratio images could be calculated via pixel-by pixel intensity ratios of the ‘factory’ and ‘field’ images. These ‘difference’ images are highly likely to highlight any changes to the module that occurred during shipment, e.g. because of rough handling. Image processing algorithms were run on each of the difference/ratio images to calculate metrics for cracks, series resistance, cell interconnects and carrier recombination defects. Each metric has a predetermined threshold above which the module would be deemed defective and not fit for installation.

### Example 2

Ten years after a module was installed in a solar farm 1216, its electrical power output dropped below the warrantied value as calculated from its original value allowing for a 0.8% drop per annum. The solar farm service staff removed and replaced the module and, as per the requirements of the warranty conditions of the manufacturer 1210, the defective module was sent to a module autopsy lab 1218 to identify the cause of failure and, if possible, identify the entity at fault. Using a line-scanning EL/PL inspection apparatus and an I-V power test unit, autopsy lab staff generated the following data: (i) line-scanning PL image; (ii) line-scanning EL image; (iii) I-V curve; (iv) time and date of test; (v) operator ID; (vi) autopsy lab ID; (vii) module ID; (viii) crack metrics from processed EL and PL images; (ix) series resistance metrics from processed EL and PL images; (x) cell interconnect metrics from processed EL and PL images; and (xi) carrier recombination defect metrics from processed EL and PL images.

The I-V test data confirmed that the module was generating lower than expected power. Inspection with the line-scanning EL/PL inspection apparatus identified a number of regions in several cells that were electrically isolated, probably due to cracks. These regions appeared relatively dark in the EL image because no current could be pushed into them, and were automatically detected and reported by series resistance and cell interconnect algorithms. The PL image revealed a number of cracks that appeared to be responsible for these isolated regions, with the cracks automatically detected and reported as quantitative metrics by a crack detection algorithm. At this point the module autopsy lab 1218 could confidently report that the module failure was due to cracking in several of the cells, although no entity could yet be identified as the one likely to be at fault. The test data 1408 from the module autopsy lab was then uploaded to the service provider 1300 to compare the recently measured data with that acquired prior to installation and at the module factory.

Several ‘difference’ images, or alternatively ‘ratio’ images, were calculated by a computing device 1402 of the service provider 1300, as follows: (A) PL image (autopsy lab) versus PL image (factory); (B) EL image (autopsy lab) versus EL image (factory); (C) PL image (autopsy lab) versus PL image (pre-installation); and (D) EL image (autopsy lab) versus EL image (pre-installation). In this case it was found that none of the cracks were present before installation, or in the newly manufactured module at the factory. The solar farm operator 1216 thus concluded that the cracks were not the fault of the manufacturer 1210 or the transporter 1212, and therefore a warranty claim was not appropriate. Instead, it was likely the cracks had been caused by rough handling during installation or service/maintenance, or by a recent hailstorm. After the solar farm operator 1216 provided the relevant results to the project owner 1224, the project owner eventually claimed the cost of module replacement with insurance. The insurance entity 1222 could, if required, request its own copy of the results from the service provider 1300.

### Example 3

A standards and quality assurance agency 1228 engaged a data analytics company to obtain and analyse module data 1408 from the service provider 1300 for all modules of a specific model number from a specific manufacturer that had been on the market for two years, with 20,000,000 units already installed in Europe or Australia. The manufacturer 1210 had set specific ‘pass/fail’ thresholds for the following metrics based on processed EL and PL images acquired with an in-factory line-scanning inspection apparatus: (i) crack metrics; (ii) series resistance metrics; (iii) cell interconnect metrics; and (iv) carrier recombination defect metrics. In each case the pass/fail threshold was set relatively high, because otherwise the reject rate would have been uneconomically high since the manufacturer 1210 had neither the budget nor the expertise to reduce the incidence of the various defects to close to zero. There was concern in the market that the levels of defects being allowed through by the manufacturer 1210 might result in an unacceptably high incidence of module failure during their service life.

Accordingly, the analytics company gathered all available data for these modules, including data from factory testing, pre-installation testing and failed module autopsy reports. The analytics company firstly identified that there were three primary causes of failure in modules that had been sent to module autopsy labs: (i) cell interconnect issues had led to electrical isolation issues and outright module failure in some modules installed in Australia, and much less commonly in modules installed in Europe; (ii) a relatively high level of carrier recombination defects were present in modules that had lower than expected power output but were not failing completely, in both Australia and Europe; and (iii) a lesser number of modules had cracks and other failure modes presumably resulting from handling incidents, hailstorms or other ‘acts of God’.

A deeper analysis including comparison of module autopsy lab, factory and pre-installation test results provided further useful information. Firstly, it was observed that the cell interconnect issues found in the modules that had failed mainly in Australia were not present prior to installation. This suggested a systematic failure mode caused by an in-factory materials or processing issue, exacerbated by the higher temperatures at Australian solar installations. Secondly, it was observed that the carrier recombination defects were not present prior to installation and were largely confined to the outer portions of cells at the edges of modules. This is consistent with chemical reactions in those cells caused by water ingress at the module edges, which is again suggestive of a materials or processing fault in the module manufacturing.

Consequently the module manufacturer 1210 was held to be at fault and therefore responsible for the replacement of all modules of this model number that failed. The manufacturer undertook to provide a store of replacement modules to project owners 1224 and also to investigate the causes of these systematic failure modes. Ultimately the failure modes were remedied by using alternative suppliers of critical materials such as the module edge sealant, and by modifying the soldering process of the cell interconnects.

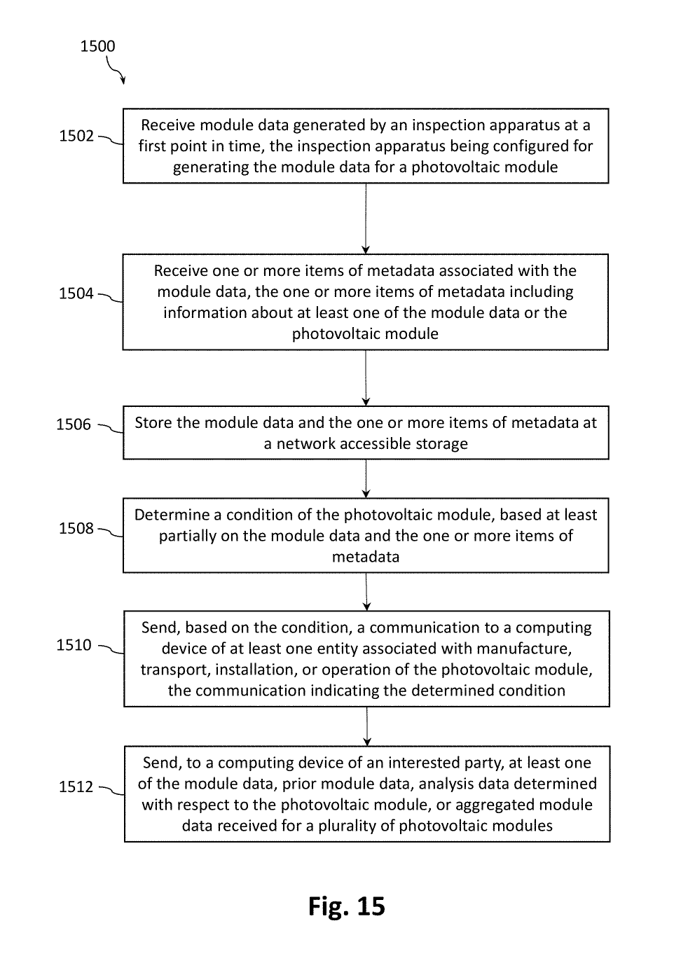

FIGS. 15-19 are flow diagrams illustrating example processes according to some implementations. The processes are illustrated as collections of blocks in logical flow diagrams, which represent a sequence of operations, some or all of which may be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation. Any number of the described blocks can be combined in any order and/or in parallel to implement the process, or alternative processes, and not all of the blocks need be executed. For discussion purposes, the processes are described with reference to the environments, frameworks and systems described in the examples herein, although the processes may be implemented in a wide variety of other environments, frameworks and systems.

FIG. 15 is a flow diagram illustrating an example process 1500 for determining conditions of modules over time according to some implementations. In some examples the process 1500 may be executed by at least one of the service computing devices 1402 or some other suitable computing device.

At 1502, a computing device may receive module data generated by an inspection apparatus at a first point in time, wherein the inspection apparatus is configured for generating the module data for a photovoltaic module. The module data may for example be received from a module inspection apparatus and/or a client computing device of an entity that manufactures, transports, installs or operates modules, or that examines failed modules.

At 1504, the computing device may receive one or more items of metadata associated with the module data, the one or more items of metadata including information about at least one of the module data or the photovoltaic module. The metadata may for example include information about module ID, tests performed, manufacturer information, transporter information, installer information, operator information or the like.

At 1506, the computing device may store the module data and the one or more items of metadata at a network accessible storage. Module data received for the module at a plurality of different points in time may be stored for instance at a network storage location to enable analysis and determination of a condition of the module at the different points in time.

At 1508, the computing device may determine a condition of the photovoltaic module, based at least partially on the module data and the one or more items of metadata. For example the computing device may determine the condition of the photovoltaic module by comparing the module data with prior module data generated for the photovoltaic module at an earlier time. Further, the computing device may determine, based on the condition, at least one of: a grade for the photovoltaic module; whether the photovoltaic module has a fault; whether the photovoltaic module is likely to develop a fault; or a cause of a fault in the photovoltaic module. Additionally, as another example, the computing device may receive additional module data generated at a second point in time by the same inspection apparatus or a different inspection apparatus, and the computing device may determine the condition of the photovoltaic module at the second point in time based at least partially on comparing the module data from the first point in time with the additional module data.

At 1510, the computing device may send, based on the condition, a communication to a computing device of at least one entity associated with manufacture, transport, installation, operation or examination of the photovoltaic module, the communication indicating the determined condition.

At 1512, the computing device may send, to a computing device of an interested party, at least one of the module data, prior module data, analysis data determined with respect to the photovoltaic module, or aggregated module data received for a plurality of photovoltaic modules.

As mentioned previously, module data may for example be generated by an inspection apparatus 500, 600, 700, 800 or 900. In some implementations an inspection apparatus 500-900 may be under the control of a computing device 510, the terminal 512 or other computing device. That is, a computing device may operate some or all of the camera 502, light source 514, power supply 302 and scanning mechanism 508, as well as various optional components such as a light source 520 and camera 522 for optical imaging, a thermal imaging camera 704, a sunlight simulator 904 and associated power supply 906 and power monitoring unit 908, and various adjustable optical components such as filters and mirrors that may be present.

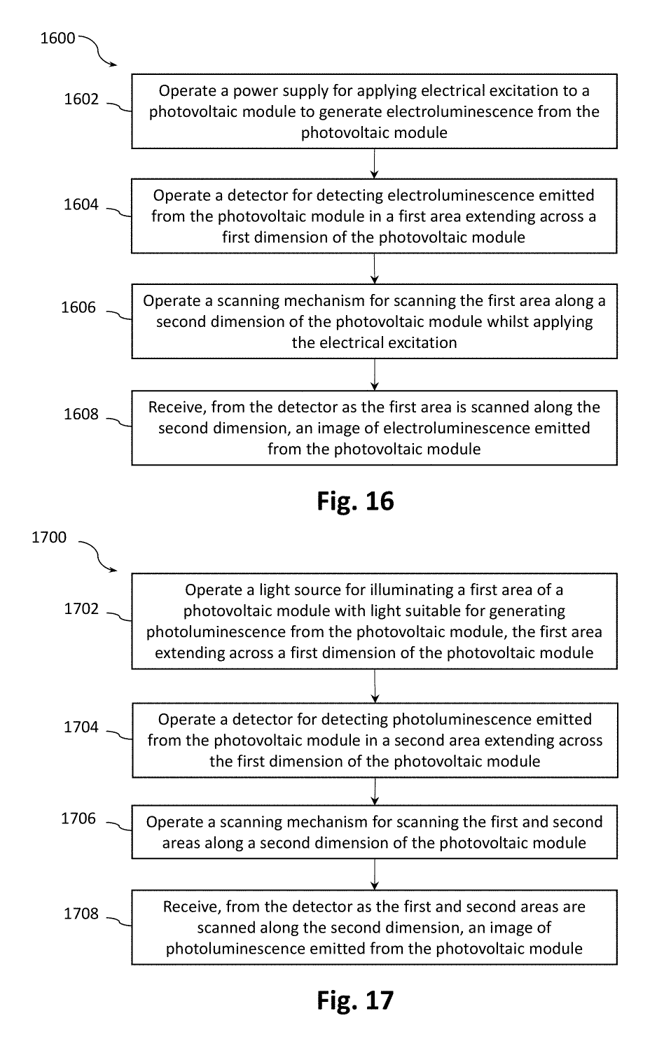

FIGS. 16-19 are flow diagrams illustrating example processes 1600, 1700, 1800 and 1900 for generating module data according to some implementations. In some examples, each of the processes 1600-1900 may be executed by a computing device 510 or other suitable computing devices.

Turning firstly to the example process 1600 illustrated in FIG. 16, at 1602 a computing device may operate a power supply for applying electrical excitation to a photovoltaic module to generate electroluminescence from the photovoltaic module. At 1604, the computing device may operate a detector for detecting electroluminescence emitted from the photovoltaic module in a first area extending across a first dimension of the photovoltaic module. At 1606, the computing device may operate a scanning mechanism for scanning the first area along a second dimension of the photovoltaic module whilst applying the electrical excitation. At 1608, the computing device may receive, from the detector as the first area is scanned along the second dimension, an image of electroluminescence emitted from the photovoltaic module.

Turning now to the example process 1700 illustrated in FIG. 17, at 1702 a computing device may operate a light source for illuminating a first area of a photovoltaic module with light suitable for generating photoluminescence from the photovoltaic module, the first area extending across a first dimension of the photovoltaic module. At 1704, the computing device may operate a detector for detecting photoluminescence emitted from the photovoltaic module in a second area extending across the first dimension of the photovoltaic module. At 1706, the computing device may operate a scanning mechanism for scanning the first and second areas along a second dimension of the photovoltaic module. At 1708, the computing device may receive, from the detector as the first and second areas are scanned along the second dimension, an image of photoluminescence emitted from the photovoltaic module.

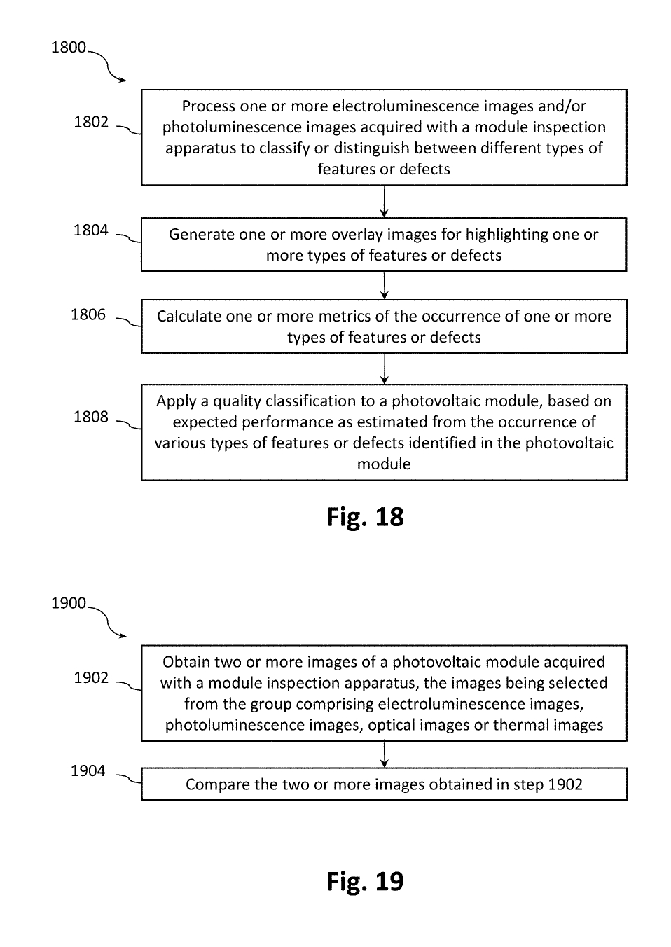

Turning now to the example process 1800 illustrated in FIG. 18, at 1802 a computer may process one or more electroluminescence images and/or photoluminescence images acquired with a module inspection apparatus to classify or distinguish between different types of features or defects. At 1804 the computer may generate one or more overlay images for highlighting one or more types of features or defects. At 1806 the computer may calculate one or more metrics of the occurrence of one or more types of features or defects. At 1808 the computer may apply a quality classification to a photovoltaic module, based on expected performance as estimated from the occurrence of various types of features or defects identified in the photovoltaic module.

Turning now to the example process 1900 illustrated in FIG. 19, at 1902 a computer may obtain two or more images of a photovoltaic module acquired with a module inspection apparatus, the images being selected from the group comprising electroluminescence images, photoluminescence images, optical images or thermal images. At step 1904 the computer may compare the two or more images obtained in step 1902.

The example processes described herein are only examples of processes provided for discussion purposes. Numerous other variations will be apparent to those of skill in the art in light of the disclosure herein. Further, while the disclosure herein sets forth several examples of suitable frameworks, architectures, and environments for executing the processes, the implementations herein are not limited to the particular examples shown and discussed. Furthermore, this disclosure provides various example implementations, as described and as illustrated in the drawings. However, this disclosure is not limited to the implementations described and illustrated herein, but can extend to other implementations, as would be known or as would become known to those skilled in the art.

Various instructions, processes, and techniques described herein may be considered in the general context of computer-executable instructions, such as program modules stored on computer-readable media, and executed by the processor(s) herein. Generally, program modules include routines, programs, objects, components, data structures, executable code, etc., for performing particular tasks or implementing particular abstract data types. These program modules and the like may be executed as native code or may be downloaded and executed, such as in a virtual machine or other just-in-time compilation execution environment. Typically, the functionality of the program modules may be combined or distributed as desired in various implementations. An implementation of these modules and techniques may be stored on computer storage media or transmitted across some form of communication media. Thus, the index arrangement herein may be implemented on physical hardware, may be used in virtual implementations, may be used as part of overall deduplication system on either physical or virtual machine, and/or may be as a component for other deduplication implementations (e.g. SAN) or in some non-deduplication environments, such as large scale memory indexing.

Although the invention has been described primarily in terms of silicon cell-based modules, the principles of the invention are not limited to this type of module. In particular, PL and EL imaging techniques can generally be applied to inspecting modules based on materials other than silicon by selecting light sources with suitable wavelength bands and illumination intensities, and cameras with suitable sensitivity and detection bands. For thin film modules based on direct bandgap semiconductors such as cadmium telluride, luminescence imaging techniques may well be easier to apply because of the often much greater luminescence efficiency of these materials compared to silicon.

Although the present invention has been described with particular reference to certain preferred embodiments thereof, variations and modifications of the present invention can be effected within the spirit and scope of the following claims.

1-16. (canceled)

17. A system for inspecting a photovoltaic module, said system comprising:
a power supply for applying electrical excitation to a photovoltaic module to generate electroluminescence from said photovoltaic module;
a light source for illuminating a second area of said photovoltaic module with light suitable for generating photoluminescence from said photovoltaic module;
a detector for detecting photoluminescence emitted from a first area of said photovoltaic module;
a scanning mechanism for scanning said first and second areas along said photovoltaic module; and
one or more computing devices programmed by executable instructions to:
receive, from said detector as said first and second areas are scanned along said photovoltaic module, an image of photoluminescence emitted from at least a portion of said photovoltaic module;
receive an image of electroluminescence emitted from at least a portion of said photovoltaic module; and
compare two or more images of electroluminescence or photoluminescence to detect or highlight defects or other features in said photovoltaic module.

18. The system according to claim 17, wherein said system is configured such that, in use, said first and second areas are at least partially overlapping.

19. The system according to claim 17, wherein said detector comprises a line camera or a TDI camera.

20. The system according to claim 17, wherein said detector comprises a contact imaging sensor.

21. The system according to claim 17, wherein said scanning mechanism comprises a mechanism for moving said photovoltaic module.

22. The system according to claim 17, wherein said scanning mechanism comprises a mechanism for moving said detector and/or said light source.

23. The system according to claim 17, wherein said scanning mechanism comprises an optical element for redirecting said photoluminescence emitted from said first area to said detector, said optical element being adapted to move along said photovoltaic module while said detector and said photovoltaic module remain stationary.

24. The system according to claim 23, wherein said scanning mechanism is configured such that the optical path length between said first area and said detector remains substantially constant as said first and second areas are scanned along said photovoltaic module.

25. (canceled)

26. The system according to claim 17, wherein said system is configured to acquire I-V test data from said photovoltaic module.

27. The system according to claim 17, wherein said system is configured to acquire an optical image of at least a portion of said photovoltaic module.

28. The system according to claim 17, wherein said system is configured to acquire an image of thermal radiation emitted from at least a portion of said photovoltaic module as a result of the application of electrical excitation to said photovoltaic module.

29. The system according to claim 17, wherein said one or more computing devices are programmed by executable instructions to process one or more photoluminescence images and/or electroluminescence images acquired with said system to classify or distinguish between different types of the defects or other features, or generate one or more overlay images for highlighting one or more types of the defects or other features, or calculate one or more metrics of the occurrence of one or more types of the defects or other features, or apply a quality classification to said photovoltaic module, based on expected performance as estimated from the occurrence of various types of the defects or other features identified in said photovoltaic module.

30. The system according to claim 17, wherein said one or more computing devices are programmed by executable instructions to compare two or more images of said photovoltaic module acquired with said system, said images being selected from the group comprising electroluminescence images, photoluminescence images, optical images or thermal images.

31-42. (canceled)

43. A method for inspecting a photovoltaic module, said method comprising the steps of:
applying electrical excitation to said photovoltaic module to generate electroluminescence from said photovoltaic module;
illuminating a second area of said photovoltaic module with light suitable for generating photoluminescence from said photovoltaic module;
detecting, with a detector, photoluminescence emitted from a first area of said photovoltaic module;
scanning said first and second areas along said photovoltaic module;
receiving, from said detector as said first and second areas are scanned along said photovoltaic module, an image of photoluminescence emitted from at least a portion of said photovoltaic module;
receiving an image of electroluminescence emitted from at least a portion of said photovoltaic module; and
comparing two or more images of electroluminescence or photoluminescence to detect or highlight defects or other features in said photovoltaic module.

44. The method according to claim 43, wherein said first and second areas are at least partially overlapping.

45. The method according to claim 43, wherein the step of scanning said first and second areas comprises moving said photovoltaic module.

46. The method according to claim 43, wherein the step of scanning said first and second areas comprises moving said detector and/or said light source.

47. The method according to claim 43, wherein the step of scanning said first and second areas comprises moving an optical element that redirects said photoluminescence emitted from said first area to said detector while said detector and said photovoltaic module remain stationary.

48. The method according to claim 47, wherein the optical path length between said first area and said detector remains substantially constant as said first and second areas are scanned along said photovoltaic module.

49. (canceled)

50. The method according to claim 43, further comprising the step of acquiring I-V test data from said photovoltaic module.

51. The method according to claim 43, further comprising the step of acquiring an optical image of at least a portion of said photovoltaic module.

52. The method according to claim 43, further comprising the step of acquiring an image of thermal radiation emitted from at least a portion of said photovoltaic module as a result of the application of electrical excitation to said photovoltaic module.

53. The method according to claim 43, further comprising the step of processing one or more photoluminescence images and/or electroluminescence images acquired from said photovoltaic module, to classify or distinguish between different types of the defects or other features, or generate one or more overlay images for highlighting one or more types of the defects or other features, or calculate one or more metrics of the occurrence of one or more types of the defects or other features, or apply a quality classification to said photovoltaic module, based on expected performance as estimated from the occurrence of various types of the defects or other features identified in said photovoltaic module.

54. The method according to claim 43, further comprising the step of comparing two or more images acquired from said photovoltaic module, said images being selected from the group comprising electroluminescence images, photoluminescence images, optical images or thermal images.

55. The system according to claim 17, wherein said one or more computing devices are programmed by the executable instructions to compare an image of electroluminescence and an image of photoluminescence.

56. The system according to claim 17, wherein said system is configured to receive said image of electroluminescence from said detector as said first area is scanned along said photovoltaic module.

57. The system according to claim 56, further comprising one or more temperature sensors for monitoring the temperature of said photovoltaic module in the vicinity of said first area as said first area is being scanned along said photovoltaic module, for enabling a temperature correction to be applied to the electroluminescence signal detected by said detector.

58. The method according to claim 43, wherein the step of comparing two or more images of electroluminescence or photoluminescence comprises comparing an image of electroluminescence and an image of photoluminescence.

59. The method according to claim 43, wherein said image of electroluminescence is received from said detector as said first area is scanned along said photovoltaic module.

60. The method according to claim 59, further comprising steps of:
monitoring the temperature of said photovoltaic module in the vicinity of said first area as said first area is being scanned along said photovoltaic module; and
applying a temperature correction to the electroluminescence signal detected by said detector.
